(19) United States
(12) Patent Application Publication
(10) Pub. No.: US 2004/0110681 A1
(43) Pub. Date: Jun. 10, 2004

(54) Method to identify targeting molecules

(76) Inventors: Xiao-Min Fan, San Diego, CA (US); Elias Lazarides, San Diego, CA (US); Catherine M. Woods, La Jolla, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/637,718

(22) Filed: Aug. 8, 2003

Related U.S. Application Data
(60) Provisional application No. 60/402,372, filed on Aug. 8, 2002.
(60) Provisional application No. 60/470,927, filed on May 14, 2003.

(51) Int. Cl. A61K 39/395; A61K 49/00; A61K 38/17

(52) U.S. Cl. 514/12; 424/130.1

(57) ABSTRACT

Methods for validating the in vivo targeting specificity of compounds according to pharmacokinetic criteria are described. An improved method to generate a library of peptides with reduced complexity and enhanced candidate compound membership comprises expressing oligonucleotides encoding cyclic peptides with randomized coding sequences representing the non-bridging amino acids. Also described is determination of structure activity relationships using kinetic technique using in vivo panning. Design of targeting compounds with appropriate space/charge/hydrophobicity conformations can be based on the results of these kinetic structure activity relationship (kSAR) determinations. In addition, compositions resulting from this methodology are useful in treating neuroinflammatory and lung disorders, and disorders involving disseminated blood cell coagulopathy interactions with vascular endothelium.

### CROSS-REFERENCE TO RELATED APPLICATIONS

This application claims priority under 35 U.S.C. § 119(e) to U.S. Serial Nos. 60/402,372 filed Aug. 8, 2002 and 60/470,927 filed May 14, 2003. The contents of these applications are incorporated herein by reference.

### TECHNICAL FIELD

The invention relates to applying appropriate criteria for identifying molecules that have organ or tissue specificity for use as drugs and/or in targeting therapeutic or diagnostic compounds to desired locations in vivo. More specifically, it concerns a method for establishing the targeting specificity of these molecules and the use of this method to rapidly generate an optimized ligand. A second major aspect of the invention relates to compositions consisting of a brain targeting ligand which has therapeutic utility in neuroinflammatory disorders.

### BACKGROUND ART

Panning combinatorial libraries in vivo to identify molecules that selectively target tumors or other specific organs has been described. For example, PCT publications WO 97/10507; WO 98/10795; and WO 99/1339 describe using phage display techniques to identify peptides that bind selectively to tumors. In the techniques described, phage-based libraries of random sequence peptides were administered to mice bearing tumors and phage retained within various organs were then analyzed for the peptide sequences expressed by the phage to identify those sequences that selectively accumulate in the target organ (in this case tumors) compared to other reference organs. Specificity for tumors was said to reach 10-fold after three rounds of selection in vivo.

Targeting to specific organs using similar techniques was described in U.S. Pat. Nos. 5,622,699 and 6,068,829, the contents of which are incorporated herein by reference. Specificity ratios for certain peptides were found as high as 8 or 9 for the brain as compared to the kidney for certain peptides. The evaluation of specificity described in this prior art was determined in a short interval (≦10 minutes) after intravenous administration of the test phage when the plasma concentrations would be expected to be maximal, without regard to the innate pharmacokinetic behaviour of the phage platform itself. As will be presented in this invention, an important improvement in the in vivo panning method is the use of phage vectors with an appropriately short half-life to allow rapid elimination of non binding phage and an improved signal to noise of target site binders.

Furthermore, it has now been found that simply obtaining a “specificity ratio” - i.e., the ratio of accumulation of a peptide within the target compared to accumulation in a reference organ of this magnitude within a short interval after administration is insufficient for identifying a compound as a useful targeting agent, since there are inherent differences in the biodistribution of particulates themselves (such as phage particles), independent of the displayed peptides, between different organs due to inherent differences that exist between organs in relative vascularity and hemodynamics. In addition to exhibiting a desired targeting ratio for the target organ or tissue as compared to other organs or blood, the candidate molecule must have a satisfactory “selectivity index”—i.e., accumulation in the target organ as compared to other non-selective compounds. Furthermore, its clearance from the target organ must be delayed as compared to its clearance from blood and other organs and its clearance as compared to unrelated molecules in the target organ must be substantially slower. Finally the targeting pharmacokinetic profile should be dose responsive.

Thus, as will be described below, appropriate pharmacokinetic data are required to validate the ability of a molecule to target specific tissues, organs or sites in vivo. Once validated in this way, the molecule can be subjected to novel methods to explore the effect of variations of its molecular features and these results employed to design additional molecules with improved targeting ability. To our knowledge, this represents a substantial improvement over previously described methods of applying in vivo display methodology to identify novel targeting ligands. U.S. Pat. No. 6,399,575 described the use of longer circulation times than described initially by U.S. Pat. Nos. 5,622,699 and 6,068,829, and PCT publications WO 97/10507; WO 98/10795; and WO 99/1339, but this was to increase the likelihood of transfer across the blood brain barrier, and not as a means of validating the targeting ability and specificity of a given displayed ligand. Furthermore, these studies all described use of filamentous phage systems most of which have long plasma half-lives and exhibit relatively high non-specific binding which limits their utility to detect specific binding with lower affinity ligands (e.g., Yip, Y. L., et al., J. Immunol. Methods (1999) 225:171-178).

The use of structure activity relationships (SAR) to optimize in vitro binding to a receptor or other biological activities is widely used in the pharmaceutical industry by medicinal and peptide chemists to define an optimal pharmacophore. This approach has been demonstrated in a number of instances for peptide ligands. For example, the rational design of RGD peptides that bind selectively to the αᵥβ₃ integrin compared to other integrin family members for the purpose of improving tumor targeting properties has been described by Gurrath, M., et al., Eur. J. Biochem. (1992) 210:911-921; and by Haubner, R., et al., J. Nuclear Med. (2001) 42:326-336. These peptides bind to a binding site on αᵥβ₃ integrin that is distinct from other binding sites that exist on the αᵥβ₃ integrin molecule for other ligands, such as the Adam-23 binding site described by Cal, S., et al., Mol. Biol. Cell (2000) 11:1457-1469. Additional examples of the progression from general RGD sequence with broad specificity to a variety of RGD-binding integrin receptor family members, to a constrained peptide with higher affinity and improved specificity to a single family member, to therapeutics are found in Blackburn, B. K., et al., J. Med. Chem. (1997) 40:717-729; Abraham, D. G., et al., Mol. Pharmacol. (1997) 52:227-236; Egbertson, M. S., et al., J. Med. Chem. (1994) 37:2537-2551.

U.S. Pat. Nos. 5,767,071 and 5,780,426 describe 7-mer and 5-mer cyclic peptide inhibitors of αᵥβ₃. These compounds are based on extrapolation from the RGD motif and include the cyclic peptide RCGGDSMCY. Additional peptides are also described. PCT publication WO 01/51508 published July 19, 2001 describes the design of peptide inhibitors of LFA-1/ICAM-1 interaction. These interactions are described in detail by Stanley, P., et al., Biochem. J. (2000) 351:79-86, and the transfer of the structural characteristics to small molecules is described by Gadek, T. R., et al., Science (2002) 295:1086-1089. Jackson, D. Y., et al., J Med Chem. (1997) 40:3359-3368 describe a similar approach to design small peptide antagonists that mimic the structural characteristics of the VCAM-1 binding site for the αᵥβ₁ integrin. A general approach to preparing peptidomimetics based on successful peptide activity is described by Hruby, B. J., Nature Rev. (2002) 1:847-858. Other reports of preparation of peptidomimetics include that of Abraham, D. G., et al., Mol. Pharmacol. (1997) 52:227-236; Blackburn, B. K., et al., J. Med Chem (1997) 49:717-729 and of Bednar, R. A., et al., J. Pharmacol. Exp. Thera. (1998) 285:1317-1326. By applying the techniques of the present invention to identify, validate and explore the space/charge/hydrophobocity electronic contour of molecules that target specific tissues or organs, additional molecules with these desirable properties can be designed, using these or other art-known methods.

The above examples represent antagonists of the integrin superfamily whose utility is designed to block the blood cell-vascular interactions that underlie responses to intrinsic (e.g., pathological angiogenesis, thrombosis, atherosclerosis or autoimmune responses) or extrinsic (infections, wounds) pathologies. This family of ligand mimetics provides a well characterized example of how subtle differences in the presentation of key chemical elements can evolve a low affinity ligand with broad specificity to multiple family members to a ligand with a high affinity and high degree of specificity to one family member. Further, studies within this family have illustrated the concept that the final optimized pharmacophore that best fits the binding pocket may bear no direct homology to the contact residues that comprise the binding site of the native protein ligand (e.g., Gadek, T. R., et al., Science (2002) 295:1086-1089 and Jackson, D. Y., et al., J. Med. Chem. (1997)40:3359-3368).

Both platelets and leukocytes play a key role in the regulation of inflammation. The activation of any of these cell types and the subsequent interaction with the endothelial lining of blood vessels is a first step in the inflammatory response. Capture and binding of leukocytes and platelets to activated endothelium in an inflamed tissue under normal blood flow involves multiple interactions between receptors expressed on the circulating blood cell and members of the integrin, selectin and immunoglobulin cell adhesion molecules (CAM) superfamilies. These interactions are typically controlled by activation of surface integrins or rapid surface deployment of CAMs, such as P-selectin, in response to local chemokine gradients on the circulatory cell type, and marked upregulation of expression of CAMs at the endothelium of the inflamed site (Hynes, R. O., Cell (2002) 110:673-687; Springer, T. A., Annu Rev Physiol. (1995) 57:827-872; von Andrian, U. H., N. Engl. J. Med. (2000) 343: 1020-1034). The generally accepted sequence of events is that the initial rolling and tethering of platelets or leukocytes under conditions of flow is mediated by the selecting, and subsequent firm adhesion is mediated by the integrin receptor interactions with their cognate CAMs within the post capillary venules, most notably, ICAM-1, VCAM-1 and MAdCAM (Carlos, T. M., and Harlan, J. M., Blood (1994) 84:2068-101; Springer, T. A., Annu Rev Physiol. (1995) 57:827-872; von Andrian, U. H., N. Engl. J. Med. (2000) 343: 1020-1034). The striking feature of these interactions is that a given integrin can have multiple binding sites for multiple CAMS, that impact not only binding and extravasation to the inflamed site, but also effector functions that drive subsequent immune responses. For example, while the binding of leukocytes to endothelial ICAM-1 plays a key role in adhesion to the vasculature of an inflamed tissue, multiple interactions between ICAM-1 and its LFA-1 receptor between leukocytes are also the basis of the formation of the “immunological synapse” in homotypic interactions (Gottlieb, A. B., et al., Arch Dermatol (2002) 138:591-600). Likewise platelet integrins exhibit a multiplicity of interactions that regulate both thrombotic events as well as the pro-inflammatory response of the activated platelet (Plow, E. F. and Ginsberg, M. H. (2000) in Hoffman, R., et al. (Eds.) Hematology: Basic Principles and Practice, 3^{rd} Ed., Churchill Livingstone, New York, N.Y., pp 1741-1752; McEver, R. P., Thromb. Haemost. (2001) 86:746-756). The major challenge in developing therapies that antagonize these reactions is to determine which aspect (i.e., which specific interaction or set of binding sites) most critically impacts clinical aspects of disease.

Emerging evidence further suggests that antagonists developed in vitro by rational screening against individual integrin-ligands interactions at the molecular or cellular level outside the context of the relevant tissue of a given disease are not necessarily predictive of clinical efficacy, since the interaction is artificially analyzed outside the context of the multiplicity of interactions that are specific to a given disorder. One example of the poor predictiveness of optimizing for in vitro binding affinities for in vivo efficacy is described by Nielsen, U. B., et al., Cancer Res. (2000) 60:6334-6440. Another striking example of the poor predictiveness of simple ex vivo assays of secondary effects in vivo is the effect of gpIIb/IIIa inhibitors on platelet-platelet interactions with arterial vessels during acute thrombosis compared to potential adverse effects of these agents during inflammatory conditions on the venous side of the vasculature (Bhatt, D. L., and Topol, E. J., Nature Reviews Drug Disc. (2002) 2:15-28; Bombeli, T., et al., J. Exp. Med. (1998) 187: 329-339). Likewise, many immune modulators designed to block leukocyte adhesion and extravasation may show efficacy in peripheral inflammatory disorders, but have had limited effect on neuroinflammatory disorders because of the differing architecture and the nature of the integrated immune response at the blood brain barrier (Davson, H, Segal, M. B., (1998) Physiology of the CSF and blood-brain-barriers. CRC press, Boca Raton; Miller, D. W., J. Neurovirol. (1999) 5:570-578). As will be described below, the present invention can identify ligands that recognize the relevant endothelial binding sites as they exist in vivo and may not necessarily be predicted by rational in vitro screening methods currently employed by those skilled in the art. Since the path of taking a lead ligand with broad specificity to several receptor family members and evolving it to a molecule with high affinity/specificity to the receptor of choice taken by practitioners of the art involves structure activity analysis, the ability to perform structure activity analysis in vivo is a novel approach to define specific therapeutic modalities using the specificity of tissue biodistribution as an assay.

The methods of the invention facilitate the discovery of peptidic ligands and ligand mimetic molecules. In one instance, these peptides are useful for the targeting of a variety of therapeutic molecules by direct coupling to a drug entity itself or to a delivery vehicle that encapsulates incorporates or binds the drug. Such delivery vehicles might be liposomes, nanoparticles, microspheres, linear or branched polymers, dendrimers, dextran, polylysine, polyglutamatic acid, polyaspartic acid, mixed amino acid polymers, poloxamers, polyethylene glycol, polysaccharides, lipids, glycolipids, phospholipids, neutral lipids, proteins, glycosylated proteins, hyaluronic acid, chondroitin sulfate, polyvinyl alcohol, polyvinylpyrrolidone, poly N-(2-hydroxypropyl) methacrylamide[PHMPA], polystyrene-maleic anhydride copolymer[SMA], polylactic acid, polyglycolic acid, poly(lactic acid-glycolic acid) copolymer [PLGA], cyclodextrin, cyclodextrin derivatives, methacryloylglycinamide [MAG], polyanhydrides, polyorthoesters, polycaprolactones, polycarbonates, polyfumarates, hydrogels, and other vehicles known in the art.

In the case, as in the example described below, where the peptide ligand mimetic has utility as a therapeutic agent in its own right, translation of the displayed peptide to a therapeutic may necessitate a more stable formulation to avoid the rapid renal and hepatobiliary clearance associated with many short peptides and to stabilize against proteolytic degradation. There are various well-known methods by those skilled in the art to increase the stability and/or plasma half-life of such lead molecules. One approach is to conjugate the peptide of interest to a water-soluble polymer formulated in a suitable pharmaceutical vehicle. Such polymers include but are not limited to linear or branched polymers, dendrimers, dextran, polylysine, polyglutamatic acid, polyaspartic acid, mixed amino acid polymers, poloxamers, polyethylene glycol, polysaccharides, lipids, glycolipids, phospholipids, neutral lipids, proteins, glycosylated proteins, hyaluronic acid, chondroitin sulfate, polyvinyl alcohol, polyvinylpyrrolidone, poly N-(2-hydroxypropyl)methacrylamide[PHMPA], polystyrene-maleic anhydride copolymer[SMA], polylactic acid, polyglycolic acid, poly(lactic acid-glycolic acid) copolymer [PLGA], cyclodextrin, cyclodextrin derivatives, methacryloylglycinamide [MAG], polyanhydrides, polyorthoesters, polycaprolactones, polycarbonates, polyfumarates, hydrogels, and other carrier polymers known in the art.

A second accepted method for increasing the plasma half-life and thereby exposure of the target tissue to a therapeutic peptide, is to couple the peptide to another peptidic moiety by standard molecular recombinant methodologies to generate a fusion peptide or protein with increased molecular weight, since hepatobiliary and renal clearance rate is inversely proportional to molecular weight. In addition to simple molecular weight considerations, certain proteins have inherently long plasma half-lives. Wocher, et al., J. Exp. Med (1967) 126:207 were the first to attribute the long plasma half-life of IgGs to the Fe portion of the molecule. Since then, using standard molecular methodologies to couple a polypeptide moiety to the Fe region has become a well validated method for increasing half-life of such polypeptides to allow them to become useful biological therapeutic agents (Capon, D., et al., Nature (1989) 337:525-531; U.S. Pat. No. 5,116,964; WO89/09622; U.S. Pat. No. 5,605,690).

### DISCLOSURE OF THE INVENTION

The invention, in one aspect, relates to an improved method for designing peptide candidates for specific tissue targeting. This method involves providing for randomization of translated nucleotide sequences so that a multiplicity of peptides is obtained. Thus, in one aspect, the invention is directed to a method to provide a multiplicity of peptide candidates which method comprises effecting expression of a nucleotide sequence putatively designed to encode a peptide, preferably a cyclic peptide of the formula CXₙC, optionally extended at the N- and/or C-terminus with additional codons (preferably <10 each), wherein C represents cysteine, X represents any gene encoded amino acid, and n is an integer of 2-25, wherein the nucleotide sequence encoding Xₙ is randomized. Thus, in this aspect, the nucleotide sequence “putatively” designed to encode amino acid sequence Xₙ may include codons which encode additional cysteine residues and/or may include codons which are termination codons. The resultant will be a series of cyclic peptides of varying ring size with optional N- and/or C-terminal extensions of additional amino acid sequence of one or more amino acids. By judicious choice of phage platforms, such peptide libraries can be designed to display motifs with terminal acidic or basic functionality. This method may be used to generate a set of preferred candidate compounds for use in further aspects of the invention.

In another aspect, the invention is directed to a method to validate the targeting ability of a candidate compound which specifically targets an organ or tissue which method comprises administering said compound in vivo to a model animal and determining its pharmacokinetic parameters in accordance with the following criteria; at a minimum, the compound must satisfy at least three of the first four.

The compound should have

1) a selectivity index (SI) for the target organ (i.e., the ratio of accumulation of the candidate compound in the target organ as compared to a compound known to be non-specific for accumulation in that organ) greater than 5, preferably greater than 10;

2) a specificity index (SPI) with respect to accumulation in other organs (i.e., the ratio of the SI values for the target compared to a given reference organ) with an index of greater than 5, preferably greater than 10;

3) the property that its clearance from the target tissue is slower as compared to its clearance from blood or as compared to other organs resulting in a difference of the area under the curve (AUC) of greater than 5, preferably greater than 10;

4) the property that its clearance from the target organ or tissue is retarded as compared to a nonspecific molecule and in contrast to the non-specific molecules is substantially slower than its clearance rate from blood; and

5) the optional property that its targeting pharmacokinetic profile is dose responsive.

With respect to 4), while any display platform can be used for this technology, including but not limited to filamentous phage and the T family of phage, the preferred method is to use a platform with relatively rapid clearance from blood, such as the T7 phage exemplified herein.

In another aspect, the invention is directed to a method to ascertain structure/activity relationships among compounds which contain at least one variable subunit, such as an amino acid, and wherein said activity is characterized by ability to target a specific organ or tissue which method comprises administering to a model animal one variant or a mixture of variants of said compounds wherein the variants contain certain patterns of variation of said subunit, and assessing the pharmacokinetics and tissue or organ retention characteristics of the various compounds in the mixture, preferably according to the foregoing criteria. This method enables a ranking of the chemical structural features that are necessary for targeting an organ-specific binding site as it is presented in vivo. By applying computer-aided analysis, a representative 3D model of the basic pharmacophore structure can be derived.

In still another aspect, the invention is directed to a compound having the charge/space/hydrophobicity conformational characteristics, i.e., the electronic shape, of the cyclic peptide CAGALCY in at least a portion of said molecule. Some of the compounds included within the scope of these variants are themselves cyclic peptides.

In addition, the invention relates to compounds of the formula

Z—A¹X¹X²X³X⁴A²X⁵ (1)

wherein Z represents a non-interfering substituent;

X¹-X⁵ represent independently selected amino acids; and

wherein each of A¹ and A² represents an amino acid or amino acid derivative or cyclizing moiety wherein A¹ and A² are coupled by a covalent bond, wherein said compounds specifically target at least one tissue, most preferably brain. In one embodiment, Z can represent the coat protein of a bacteriophage or a linker coupled to a drug payload of interest to be directed to the target site. The peptide can be synthesized by standard solid support methodologies known by those skilled in the art and any linker modality can be added at the amino terminus of the disclosed peptide during synthesis to facilitate coupling to the drug or therapeutic agent of choice. Such linkers can be rationally designed to include flexible spacer linkers (for example additional glycine or alanine residues, short linear aliphatic linkers), and can include additional functional groups to facilitate coupling to the desired therapeutic payload or imaging agent. Where the peptide itself is of therapeutic utility, Z can represent a variety of compositions to improve the plasma stability/plasma half-life of the peptide. These compositions include but are not limited to water soluble polymers or the carboxy terminus of proteinacious supports including, but not limited to, Fc or antibody fusion proteins or glutathione-S-transferase.

In still another aspect, the invention is directed to a subset of cyclic peptides of formula (1) having formula (2) wherein X¹-X³ are independently hydrophobic aliphatic residues, the amino acid residue at the X⁴ position is N, M or L, and the amino acid at the X⁵ position is Y or F. These compounds are of the formula

Z—A¹—X¹—X²—X³—(N/M/L)—A²—(Y/F) (2)

where Z, A¹ and A² are as defined above, and X¹-X³ are independently hydrophobic aliphatic residues, preferably A, G, L, M, V or I.

These peptides are useful as therapeutics for the treatment of neuroinflammatory disorders when formulated in a suitable pharmaceutical vehicle. More generally, these compositions are useful to treat any inflammatory disorder that involves pathological interactions of activated platelets or leukocytes with vascular endothelium. Thus, in another aspect, the invention relates to the use of compounds of formula (2) in the treatment of inflammatory disorders and to pharmaceutical compositions of the compound of formula (2).

### BRIEF DESCRIPTION OF THE DRAWINGS

FIG. 1 shows the selectivity index of the T7-A3 phage isolate for brain compared to two reference organs, lung and liver. The T7-A3 phage displays the peptide CAGALCY, that was enriched by in vivo panning to brain.

FIG. 2 shows yield of T7-CAGALCY (originally denoted T7-A3 or just A3) and control phage T7-lacZ from the brain, lung, liver, kidney, spleen and blood at different time intervals following administration (yield is defined as the ratio of the number of phage recovered from a given organ or tissue to the number of phage injected).

FIG. 3 shows the dose response of the selectivity index of T7-CAGALCY for various tissues (the ratio between the yield of T7-A3 to control phage T7-lacZ obtained from the brain and the reference tissues).

FIG. 4 shows the ability of a GST-fusion protein expressing the A3 peptide at the carboxy terminus to effectively compete with T7-A3 specific binding to brain tissue.

FIG. 5 shows selectivity index for various organs plotted for A3, the A3 variant which is Y7F, the A3 variant C6S, the A3 variant L5P and the variant lacking Y7 (ΔY7).

FIG. 6 shows graphs of frequencies of the 20 phage variants where position 5 is filled by any one of 20 amino acids in the initial phage mixture (input) compared with the frequency of sequences in phage accumulating in the target organ, brain at 30 minutes and 4 hours. One hundred and seven (107) sequences were determined in the phage pool administered (input), and 88 and 94 individual phage from the pools recovered from brain at 30 minutes or 4 hours after administration, respectively.

FIG. 7 shows the distribution among samples analyzed for amino acid at position 7 in a mixture of phage in which all 20 amino acids are provided at this position. One hundred eighty-four (184) sequences were determined in the phage pool administered (input), and 173 each of phage pools recovered from brain at 30 minutes or 4 hours after administration to assess the relative shifts in frequency within the target organ.

FIG. 8 shows a head to head comparison of the relative selectivity of the three preferred CAGAXCY variants (L5, N5 and M5) identified by the kSAR analysis presented in FIG. 6 to validate their relative targeting activity with respect to an internal standard, the control phage (SI values represent accumulation of the test phage relative to the control phage) over the six hour time period following administration of the phage.

FIG. 9 shows a minimized three dimensional (3D) structure of cyclic acetyl A3 using the PM3 Hamiltonian technique.

FIG. 10 shows a comparison between the structure of FIG. 9 and a similarly constructed structure of the cyclic acetyl A3 variant L5P.

FIG. 11 shows a comparison between the cyclic acetyl A3 structure of FIG. 9 with a comparably constructed model of a cyclic acetyl A3 variant where the alanine at position 2 is deleted.

FIG. 12 shows a comparison between the cyclic acetyl A3 model of FIG. 9 and a similarly constructed cyclic acetyl A3 variant model where alanine at position 2 is replaced by glycine.

FIG. 13 shows the effect of a fusion protein of the A3 ligand on the adhesion of platelets and platelet thrombi in the LPS-induced model of sepsis. Mean platelet count adhering to the pial vasculature in the presence of the A3-GST fusion protein compared to control GST protein and the αIIbβ3 inhibitor, Integrilin, were assessed using intravital microscopy and image analysis. Only the difference between the A3-fusion protein-treated and PBS control group is significant (P<0.0001).

### MODES OF CARRYING OUT THE INVENTION

The present invention has several aspects, leading ultimately to the identification and validation of compounds that successfully target desired tissues, organs, or, for example receptors, in vivo. A first aspect relates to validation of an initial candidate preferably, but not necessarily, selected by in vivo panning wherein the candidate has been identified by its elevated presence in a desired location after one or several rounds of selection. In in vivo panning, a mixture of compounds, generally containing a retrievable tag, is administered to an animal model and allowed to circulate for a predetermined period of time. At that time, the animal is sacrificed and the target site harvested and compounds that have accumulated at that site are recovered. Depending on the initial complexity of the library, the recovered compounds are then either analyzed directly or administered to model animals and the process is repeated as needed. After several rounds of selection, the contents of the desired site with regard to the administered compounds are analyzed and compounds that appear to accumulate at the target site are used as candidates in the method of the invention.

However, it is not necessary that the candidate compound for use in the method of the other aspects of the invention be identified in this way; any compound may be employed.

In one preferred embodiment, the candidate compounds are peptides and the retrievable tags used in the methods of the invention are phage. This has the advantage of permitting ready amplification of peptides that are harvested from particular sites.

In an improved form of this method, or, indeed, of any method wherein a multiplicity of candidate peptides is generated by expression from an encoding nucleotide sequence, the variety in the candidate library within desirable bounds can be enhanced by generating a set of cyclic peptides where the cyclic peptides are encoded by a nucleotide sequence having codons putatively corresponding to the form CXₙC where C is cysteine, X is any amino acid, and n is preferably less than 25, more preferably less than 20, more preferably less than 15, and more preferably 10 or less, but in any event at least 2 and preferably at least 4. The codons for these amino acids may be extended by an arbitrary number of codons at the N- or C-terminus upstream of the first or downstream of the second cysteine codon. The number of codons in any such extension may typically be 15 or less, preferably 10 or less. Although X is usually an amino acid codon, it may also be a termination codon. Thus, in addition to peptides of the formula CXₙC or its extended forms, a multiplicity of other cyclic forms may be generated due to the presence of cysteine codons and/or termination codons at various positions of X. The inclusion of a termination codon prior to the second encoded cysteine in combination with an upstream cysteine codon will result in cyclic peptides shorter than those indicated by the above formulation. Thus, as illustrated below, a nucleotide sequence of the form CX₁₀C yields a multiplicity of cyclic peptides including truncated peptides with free carboxy termini as a result of the degeneracy of the X codons. Because of enrichment due to successful targeting, the most frequently retrieved peptide from in vivo testing for brain targeting in the example presented below is of the form CX₄CX.

While generation of the candidate peptides in phage for in vivo panning is very convenient, with respect to the validation testing, in principle, any compound could be used and a retrievable tag employed to recover this compound and analyze it. Thus, the compounds administered might be labeled with fluorescent beads, including magnetic beads, which could then be recovered and analyzed. If additional amounts of compound are required, depending on the nature of the compound, additional compound might be supplied simply by synthesis.

In the validation method of the invention, the candidate compound must satisfy at least three of four pharmacokinetic parameters that verify its ability to target a particular site. These pharmacokinetic parameters include 1) a satisfactory selectivity index (SI); 2) a satisfactory specificity index (SPI); 3) a satisfactory measure of retention designated the area under the curve (AUC); 4) a satisfactory half-life, a measure of the clearance rate from blood or a particular organ. Preferably the compound also displays dose responsive kinetics.

The various criteria used to evaluate and validate successful targeting compounds can be defined as follows:

The “selectivity index” (SI) refers to the level of compound bound to the tissue as compared to the level of a non-specific or irrelevant compound with similar blood pharmacokinetics. Thus, if compound A is the candidate compound, and it is understood that compound B distributes nonspecifically, the ratio of A to B in a desired site at a specified time after administration is the selectivity index of compound A. In order to fulfill this criterion, compound A must have a selectivity index >5, preferably >10, preferably >20, preferably >100 at any time period after administration such as 30 minutes or 1 hour or 2 hours or 4 hours. Satisfaction of this criterion at any of these time points is sufficient. The concentration is determined by any convenient method depending on the manner in which the compound is administered. If the compound is a polypeptide and is administered by phage display, formation of plaques can be used as an index of concentration. Other methods can also be used, depending on the manner in which the compound is labeled as will be further described below.

The “specificity index” (SPI) of a compound for an organ or tissue refers to the ratio of the SI of the compound that is found in the target tissue at a specific time after administration as compared to the SI of that compound in other organs and tissues. In order to meet this criterion, the specificity index for the candidate must be >5, preferably >10, preferably >20, and more preferably >100 at any time period after administration such as 30 minutes, 1 hour, 2 hours and 4 hours after administration. Satisfaction of this criterion at any one of these time points is sufficient. With respect to the reference organ for which the value of the ratio is obtained, this is arbitrarily chosen among those organs which do not appear to harbor high concentrations of the compound. That is, in reviewing the data, typically there will be one or several organs which have noticeably elevated concentrations of the target compound and a multiplicity of organs where the concentrations are relatively similar and relatively low. Any one of these latter organs may be arbitrarily chosen as the reference organ.

The area under the curve (AUC) refers to the area beneath the curve generated when the time after administration is plotted on the x axis of a graph and the concentration of the compound in the relevant tissue or organ at these time intervals is plotted on the y axis. The AUC offers a measure of the retention of the material in a particular measured location over a chosen interval of time and provides a measure of the total exposure of the tissue of interest to a compound integrated over time. If the area under the curve is large, the compound is shown to be retained at the target organ. If, however, the compound is rapidly cleared from the location in question, the area under the curve will be small. The area under the curve may be enhanced by selective concentration at the location, or simply a reduced rate of clearance. Therefore, as taught in the present invention, both parameters need to be assessed before a conclusion of selective retention at the target site can be drawn. Thus, in order to make adequate comparisons, specific times after administration must be designated, and again, satisfaction at any one of the time points mentioned above is satisfactory.

One measure of the “clearance rate” is the “half-life” of the compound from or in blood or an organ or tissue which is defined as the time required for the concentration to be reduced to half. As this parameter is essentially a measure of rate, a specific time point need not be chosen.

The “dose responsiveness” referred to herein refers to the dependence of the measured selectively index on the dosage level. A satisfactory dose response relationship will be that wherein the selectivity index increases as the level of dosage increases to a maximal plateau, representative of saturable binding.

Thus, in order to validate the ability of a compound to target selectively an organ or tissue, at least three parameters describing its pharmacokinetic profile must be satisfied:

1) it must have a selectivity index (SI) >5;

2) it must have a specificity index (SPI) >5;

3) it must have a clearance from a target tissue or organ that is slower as compared to its clearance from blood or as compared to other organs resulting in a difference of the area under the curve (AUC) of greater than 5, preferably 10; (all of the foregoing measured at a suitable time point after administration such as 30 minutes, 1 hour, 2 hours or 4 hours independently of the parameter); and

4) the differential between the clearance rate from the target organ compared to the clearance rate from blood must be substantially slower than for a non-targeting molecule.

In addition, it is desirable, as a fifth criterion, that the selectivity index be responsive to dosage. That is, the selectivity index should increase as the level of dosage increases and exhibit hallmarks of saturable binding.

That is, three of the first four criteria must be met; the fifth criterion is optional but desirable.

Thus, in the method of the invention, a candidate compound is administered to an animal model such as a mammalian or avian model of either a normal or disease state and the criteria above determined in order to validate its status as a successful targeting molecule.

A second aspect of the invention relates to optimizing a targeting molecule with respect to its activity in vivo. Once such a molecule has been identified (by any means, not just that of the method of the invention) the kinetic technique of in vivo panning can be used to determine structure activity relationships in an efficient way. The effect of variation of certain features of the molecule can be rapidly explored using one of a number of approaches. First, it is assumed that the compound has specific structural subunits or features. If the compound is a polysaccharide, the subunit is typically an individual sugar; if the compound is a peptide, a typical subunit is an amino acid. If the compound is a synthetic organic molecule, the structural feature may be a functional group or a subset of atoms that is chosen for its spatial or charge contour characteristics. As this method defines structure activity relationships in a kinetic setting, it can be referred to as a kinetic structure activity relationship (kSAR) determination.

In the invention method, variants are constructed where one or more of the defined subunits is altered to obtain a single variation of this subunit or to obtain multiple variants thereof which can be assessed simultaneously. Thus, at one level, a single subunit may be replaced by a variant form—e.g., glycine might be replaced by alanine, or a carbonyl group may be replaced by a carboxyl group or a benzene ring replaced by a cyclohexane. At a second level, several such subunits are varied at the same time—i.e., a variant compound obtained where two or more of the subunits are each replaced by a single altered form. For example, in a peptide CGAPT, G could be replaced by S and P by V, or in the peptide C¹AGALC²Y, C¹ might be replaced by homocysteine and L by V. In a third level, a single subunit is varied but multiple compounds are simultaneously assessed where a multiplicity of replacements for the varied subunit is employed. For example, a particular subunit glycine is replaced in one molecule by alanine, in another by serine, in another by tryptophan, and so on, or a carbonyl group is replaced by a carboxylic acid in one molecule, a carboxylic ester in another molecule, an amide moiety in another, and so on. Mixtures containing each of these compounds can then be used in the kinetic in vivo panning (kSAR) method of the invention to assess their relative targeting ability. At still another level, multiple variations may be made in multiple subunits resulting in complex mixtures of compounds containing a large number variations which can be assessed simultaneously. By combining these approaches, the kinetic in vivo kSAR selection method ranks the variants and allows the preferred structural characteristics for a given targeting activity to be defined.

These approaches are illustrated below where a candidate compound validated by the pharmacokinetic criteria of the invention is altered in each of its subunits individually and the resulting variants tested for targeting ability, where individual amino acid subunits are simultaneously replaced in mixtures of compounds by the 19 alternative gene encoded amino acids, and where certain subunits are held constant but multiple variants of the remaining subunits are employed in mixtures to make these assessments. The results of such analyses lead to a series of conclusions about the required space/charge/hydrophobicity conformation of a successful targeting molecule.

The “space/charge/hydrophobicity conformation” of a molecule or of a portion of a molecule refers to the contours of the atoms and bonds forming the molecule in three-dimensional space (i.e., the space component) and the location of electron density in three-dimensional space (the charge+hydrophobicity components). A particularly important feature of the space/charge/hydrophobicity conformation is the bulk emplacement of any hydrophobic regions. Thus, for example, in the brain-specific compound CAGALCY illustrated below, the overall conformation is dictated by the ability of the peptide to attain a cyclic configuration through disulfide bonding of the two cysteines and the proximity of an aromatic moiety with a free carboxyl group provided by the Y residue. Other features of the space/charge/-hydrophobicity conformation of this molecule will be apparent from the description below. The ability to translate a predetermined space/charge/hydrophobicity conformation into a series of “small molecule” compounds is understood in the art, e.g., as set forth above and is exemplified, for example, by the generation of LFA-1 antagonists using an ICAM-1 immunoregulatory epitope as a model. A number of compounds were generated based on the space/charge conformation of this epitope which were successful in mimicking the binding of the epitope to LFA-1 (Gadek, T. R., et al., Science (2002) 295:1086-1089).

By use of varying modeling techniques, the overall space/charge/hydrophobicity conformation of any candidate molecule can be obtained and compared to unsuccessful candidates. By exploring the differences between the successful and unsuccessful candidates in terms of space/charge/hydrophobicity conformation, alternative molecules are designed which incorporate the essential features of the successful candidates while avoiding the traits which characterize the unsuccessful ones. As illustrated below, various commercially available modeling programs can be used to generate models of the successful candidate CAGALCY and the required features ascertained as described in that example. Then, using conventional techniques, alternative molecules that retain the required functional features are designed. These molecules may be peptides themselves, or may be synthetic organic molecules, as was illustrated in the article by Gadek, T. R., et al., Science (2002) 295:1086-1089, cited above. Given the unexpected nature, and unpredictability of translating in vitro characteristics to in vivo properties, a third aspect of this invention relates to applying the kSAR technique to known peptide mimetics to optimize for the required pharmaceutical properties (such as target specificity, pharmacokinetics) for in vivo functionality.

Another aspect of the invention relates to the use of cyclic peptides of formula (1), i.e., Z—A¹X¹ X² X³ X⁴ A² X⁵ and in particular the subset wherein X¹-X³ are independently aliphatic residues, X⁴ is N, M or L, and X⁵ is Y or F for targeting a drug or therapeutic agent to brain or lung, to increase exposure at the target site while minimizing exposure of other organs. As shown in the examples below, Z can represent a range of substituents without impairing the targeting ability of the parent peptide. Thus Z can represent the coat protein of a bacteriophage, and thus facilitate the targeting of a phage particle to the brain, a tag that facilitates imaging of the peptide location, or a polypeptide sequence. As will be appreciated by practitioners of the art, such peptides can be readily made by standard solid phase synthesis methods where an additional functional group can be incorporated to facilitate coupling to the carrier, drug or imaging agent of choice. Thus, in an intermediate form of formula (1), Z can represent a linker, including but not limited to additional glycine or alanine residues or a short chain aliphatic molecule, such as aminocaproic acid, to provide a flexible linker/spacer, functional groups such as amino, carboxyl, sulfhydryl, esters, aldehyde, hydroxyl, etc., to facilitate conjugation of said peptide to a drug entity or any desired a water-soluble polymer formulated in a suitable pharmaceutical vehicle. Such polymers include but are not limited to linear or branched polymers, dendrimers, dextran, polylysine, polyglutamatic acid, polyaspartic acid, mixed amino acid polymers, poloxamers, polyethylene glycol, polysaccharides, lipids, glycolipids, phospholipids, neutral lipids, proteins, glycosylated proteins, hyaluronic acid, chondroitin sulfate, polyvinyl alcohol, polyvinylpyrrolidone, poly N-(2-hydroxypropyl)methacrylamide[PHMPA], polystyrene-maleic anhydride copolymer[SMA], polylactic acid, polyglycolic acid, poly(lactic acid-glycolic acid) copolymer [PLGA], cyclodextrin, cyclodextrin derivatives, methacryloylglycinamide [MAG], polyanhydrides, polyorthoesters, polycaprolactones, polycarbonates, polyfumarates, hydrogels, and other vehicles known in the art.

In addition, the peptide structure can be modified to generate a more stable molecule. The amino terminus can be modified by electrophiles to diminish proteolytic degradation. The amino terminus can be transformed by various reactions including but not limited to alkylation, acylation, oxidation, and carbamylation The intramolecular disulfide bond can be modified or replaced to generate a more stable cyclic structure. One or both sulfur atoms could be replaced by other moieties including but not limited to alkyl, amide, ketone, hydroxyalkyl, alkene, alkyne, imine, amine, alkylamine, ether, ester, urea, carbamide, carboxamide, or carbamate.

The drug agent can include, but is not limited to any from the following groups: chemotherapeutic agents, immune modulators, 5HT agonists and antagonists, NMDA antagonists, neuralgesics including opioid analgesics, steroids, psychotropic agents and anxiolytics. Likewise a variety of imaging agents could also be coupled through a linker for diagnostic purposes. For pulmonary targeting the agents can be selected from the following non-limiting examples; vasoconstrictors, vasodilators, bronchoconstrictors, bronchodilators, anti-neoplastic agents, surfactants, steroids, antibiotic agents, antioxidants and antiproteases, or a combination thereof. Pulmonary specificity can be readily enhanced by formulation in a suitable carrier for nebulizer, dry powder inhaler or metered dose inhaler mediated delivery to the airways.

Another aspect of the invention relates use of the candidate targeting molecule, A3, predicted from the modeling of the targeting peptide and evaluation of its structural similarities or differences to known families of ligand mimetics for receptor superfamilies. Specifically, as will be shown in the examples below, an aspect of the invention relates to the use of cyclic peptides of formula (1) or the subset of formula (2), as therapeutics in the treatment of neuroinflammatory disorders when formulated in a suitable pharmaceutical vehicle. Such applications include but are not limited to hemorrhagic shock, ischemic reperfusion injury, stroke, vascular dementia, Alzheimer's Disease, multiple sclerosis, cerebral malaria, meningitis as well as other brain disorders secondary to septicemia or trauma, traumatic brain injury, cerebral edema, and inflammatory sequelae of epilepsy. Pulmonary inflammatory indications include asthma, chronic obstructive pulmonary disease, pulmonary embolism, pulmonary edema and acute lung injury. More broadly these compositions may have applications in the treatment of any inflammatory disorder that involves pathological interactions of activated platelets and platelet-complexes with vascular endothelium.

Short monomeric peptides are known to be rapidly cleared from the circulation by hepatobiliary and renal clearance. However, those skilled in the art will appreciate that there are well validated methods for extending the plasma half-life to accommodate the therapeutic window. Therefore in one embodiment, the invention includes the peptides sequences disclosed formulated on a carrier such water-soluble polymer formulated in a suitable pharmaceutical vehicle as described above. Similarly, as known by practitioners of the art, the disclosed peptides can be formulated in a variety of agents for controlled release from slowly dissolving or eroding formulations including but not limited to alginates, polylactic-co-glycolic acid [PLGA], polylactic acid, polyglycolic acid, polyanhydrides, polyorthoesters, polycaprolactones, polycarbonates, polyfumarates, liposomes, nanoparticles, microspheres, cyclodextrin, or hydrogels. Alternatively, a fusion protein expressing the peptide at the carboxy terminus such that the ligand is appropriately presented can be generated by standard molecular cloning techniques. These can include Fc domains or other antibody derivatives in a suitable pharmaceutical vehicle.

The dosage levels, routes of administration, and appropriate formulations of the compounds of the invention used as pharmaceuticals are readily optimized by practitioners. Dosage ranges are dependent on the nature of the condition, the susceptibility of the subject, and the judgment of the practitioner. Suitable subjects include human and veterinary subjects, both avian and mammalian. Routes of administration are those generally suitable for peptides or small molecules designed based on the space/charge/hydrophobicity conformation of the compounds of formula (2), and more specifically on the space/charge/hydrophobicity conformation based on compound A3—i.e., CAGALCY in cyclic form. Suitable formulations for various administration routes may be found, for example, in Remington's Pharmaceutical Sciences, latest edition, Mack Publishing Co., Easton, Pa. Routes of administration include transdermal, transmucosal, oral, intravenous, intramuscular injection, subcutaneous injection and the like. Formulations can readily be designed for any of these administration routes.

The following examples are intended to illustrate but not to limit the invention.

### EXAMPLE 1

### Isolation of a Candidate Brain-Targeting Compound

The in vivo panning techniques used in this example are those described, for example, in U.S. Pat. No. 6,068,829 referenced above and incorporated herein by reference. Briefly, in order to isolate peptides that target brain vasculature, including peptides that enable T7 phage to cross the blood-brain barrier, peptides displayed on the coat proteins of T7 phage are administered to the tail vein of murine subjects and the phage allowed to circulate for a suitable time period, typically 1 hour, to allow time for sufficient clearance of non-binding phage from the circulation for a more accurate determination of specific binding events. After that time, the mice are sacrificed and the brain tissue harvested and homogenized. Phage are recovered from the brain, amplified and re-injected for succeeding rounds of selection. After a suitable number of rounds of selection, phage present in the harvested brain tissue are recovered and sequenced. The presence of multiple phage plaques containing similar or identical sequences is evidence that the enriched sequence is successful in targeting the brain vasculature.

In this example, a “CX₁₀C-T7” phage library was constructed so that the peptides were displayed on each of the 415 copies of the 10B co-protein of the T7 phage surface.

Briefly, oligonucleotides with suitable redundancies in the genetic code were designed to encode a 12 amino acid peptide containing cysteines at the N- and C-termini. By appropriately randomizing the X₁₀ encoding sequence, additional peptides could be obtained. Thus, because the nucleotide sequence between the two encoded cysteines is random, codons would be present at various locations which encode additional cysteines and which encode termination codons. Thus, as will be seen from the results herein, the compound which showed the highest level of brain targeting was actually of the formula CX₄CX.

The oligonucleotides were gel purified and ligated to T7 (415-1) vector and mixed with packaging extract to obtain a library. The library was amplified in the BLT-5615 bacterial host strain in the presence of 1 mM IPTG.

The resulting library was purified using standard purification techniques and then used for panning studies. An aliquot of the phage library was injected into the tail veins of female FVB/N mice, allowed to circulate for varying lengths of time, before anesthetizing the mice and perfusing the organs with PBS to remove the excess unbound phage. The brains were harvested, rinsed with cold PBS, and homogenized in 500 μl PBS containing 0.5% NP-40 and 2% BSA with a Dounce homogenizer.

1.5 ml PBS was then added to bring the total volume to 2 ml and aliquots were removed for plaque assay. The remaining homogenates were amplified in BLT-5615 in the presence of 1 mM IPTG, purified and used for the next round of panning which was conducted as described for the first round set forth above.

Plaque assays were used to determine the relative amounts of phage contained in blood and brain. For the blood assays, 300 μl of blood was drawn into 300 μl of 100 mM sodium citrate before perfusion. Plasma was recovered and saved for plaque assay. The phage content was assessed by mixing aliquots of the tissue homogenate or plasma with the bacterial BLT-5615 host, then plating on agar plates.

40 μl of 40 mg/ml of X-gal in DMF was added into 3 ml of top agar in each plate. After incubation at 37° C. the number of clear plaques (test phage) to blue plaques (control T7-lacZ phage) were determined.

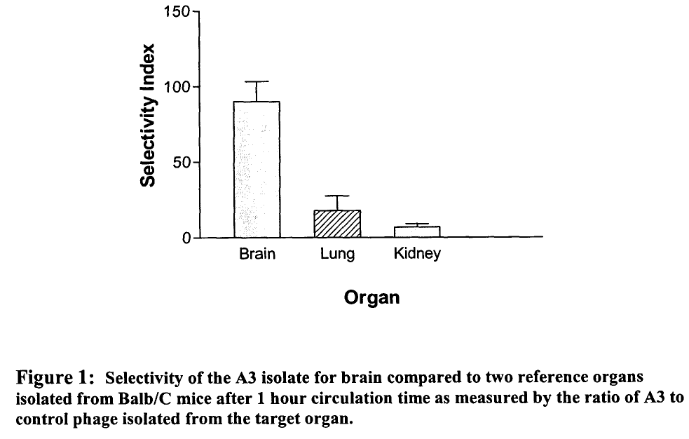

A total of 76 phage were picked from the fifth round of selection and the inserts amplified by PCR and sequenced. Of these, a total of 30 represent the sequence CAGALCY. One of the clones expressing this peptide, CAGALCY, initially designated A3, was compared in vivo to control T7-LacZ phage. A mixture of the two phage (10⁹ pfu of each phage) was injected into the tail veins of non-anesthetized BALB/c mice. After 1 hour circulation time, the mice were anesthetized, perfused with PBS containing 1% BSA, and organs were harvested and homogenized and the numbers of A3 and control T7-LacZ phage recovered from the tissue and blood were determined. FIG. 1 shows the ratio of A3 to control phage (the selectivity index) obtained in the brain compared to two reference tissues, lung and kidney (particulates can readily marginate in lung and kidney is one of the sites of clearance for small blood borne particulates). This indicates that A3, which exhibited a slightly shorter half-life than control phage targets brain selectively. Another measure of selective retention is to compare target organ to blood levels of the test phage. A3 showed a high brain to blood ratio (>>100-fold after normalization to control phage) compared to other organs.

### EXAMPLE 2

### Pharmacokinetics of CAGALCY

In this example, it is demonstrated that the peptide CAGALCY has a high selectivity index when compared to non-specific, LacZ-T7, a high specificity index for brain with respect to other organs or blood, and clears from brain much more slowly than from blood, but clears from blood substantially at the same rate as LacZ-T7. In contrast the control phage clears from brain with similar pharmacokinetics as its clearance from blood. This selectivity is dose-responsive.

A3-T7 was amplified in the host strain BLT-5615 in the presence of 1 mM IPTG and purified. T7-lacZ phage were amplified by large scale plating on LB plates. The plates were incubated at 25° C. overnight, and phage were eluted with phage extraction buffer (20 mM Tris-HCl, pH 8.0, 100 mM NaCl, 6 mM MgSO₄) overnight at 4° C. with gentle rocking. The extracted phage were pooled and purified. In the PK study, in paragraph A below, three mice were used per time point, co-injected with 1×10¹⁰ pfu of each phage. Time points were 2 min, 10 min, 30 min, 1 hr, 2 hr, 4 hr, 6 hr, 12 hr, and 24 hr.

In the dose response study, in paragraph B below, three mice were used per dose, co-injected with 1×10⁷, 1×10⁸, 1×10⁹, 1×10¹⁰, 1×10¹¹ pfu of each phage.

### A. Pharmacokinetic (PK) Analysis of T7-CAGALCY and T7-lacZ phage

1×10¹⁰ pfu of T7-CAGALCY and T7-lacZ phage were co-injected into FVB female mice. Mice were anesthetized with Avertin 2-4 minutes before the scheduled harvest time (the mice assigned to the 2 minute time-point were anesthetized prior to administration of the phage). The brain, lung, liver, kidney, blood and spleen were harvested at 2 min, 10 min, 30 min, 1 hr, 2 hr, 4 hr, 6 hr, 12 hr, and 24 hr. The phage contents in each organ were determined by plaque assay in the presence of X-gal in top agar.

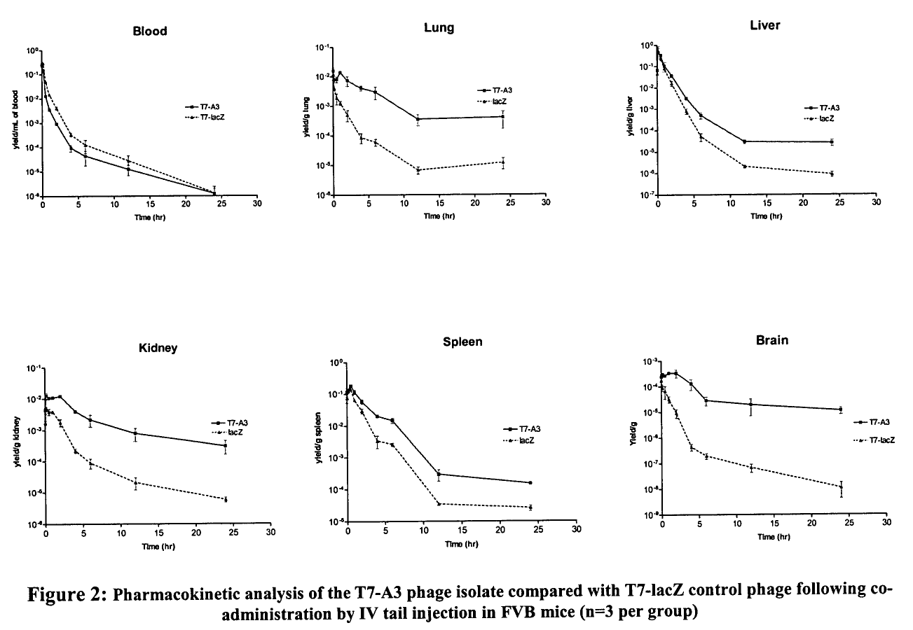

FIG. 2 shows yield/g for each organ and yield/mL obtained in blood. As shown, T7-A3 is rapidly cleared from the blood, with a half-life of 8.9 min. The T7-lacZ control phage shows a slightly longer half-life of 14.7 min. T7-lacZ was cleared faster from all the organs examined compared to the T7-A3 phage, indicating the T7-A3 phage has a greater tissue retention than the control T7-lacZ phage in these organs. However, the most marked accumulation was in brain.

The number of the T7-A3 phage recovered from the brain increased slightly over the first 2 hr, declining slowly thereafter, whereas the amount of control phage declined rapidly in the first 4 hr (alpha phase), then slowly declined between the 4 and 24 hr time points (beta phase). Thus the rate of clearance of control phage from the brain paralleled the pharmacokinetics of clearance from blood, whereas the A3 phage increased slightly over the first 1 to 2 hour period despite a two log decline in the blood concentration over this same time interval.

In the lung and kidney, the T7-A3 phage also appeared to accumulate slightly in the first 30 min, before steadily declining. In the spleen and liver, the amount of both T7-A3 and T7-lacZ phage declined steadily after the first time point (2 min). At every time point examined, more T7-A3 phage than T7-lacZ control phage were found in all organs examined in this study. However, the greatest differential retention was observed in the brain with a selectivity index (SI) of 51 at 2 hr and 280 at 4 hr. This compared to SI values ranging from 1.98 in spleen to 16.0 in lung at 2 hr, and from 4.62 in liver to 54.8 in lung at 4 hr.

When the difference of the half-lives of these two phage in blood was taken into account, the ratio of yield/g brain and yield/mL blood is much greater between the T7-A3 and T7-lacZ phage, with ratios of 250 at 2 hr and 1200 at 4 hr. Thus, the selectivity index for T7-A3 is well over 100. In the methods that have been presented previously for performing in vivo phage display, for example, PCT publications WO 97/10507; WO 98/10795; and WO 99/1339 as well as U.S. Pat. Nos. 5,622,699 and 6,068,829, the ratio of the test phage in the target organ to brain at a short time interval (5 to 10 minutes) after administration was used as a measure of selectivity. However, as demonstrated in Table 1 below, at 10 minutes there are marked differences in the amounts of the control phage platform itself recovered from different organs due to the differences in vascularity per gram tissue of different organs. An organ such as lung which is highly vascularized has a 100-fold higher content of phage than does brain at all time points following administration. Likewise liver and spleen also have a high blood pool per unit weight and in addition serve as the primary sites for clearance of foreign particulates such as phage from the circulation.

TABLE 1
Yield of control T7-lacZ and T7-A3 phage recovered from tissues
(pfu/gm) or blood at three selected time intervals after 1 ×
10¹⁰ of each phage was administered per mouse (n = 3
per group) (pfu/mL)

| | | | |
|---|---|---|---|
| Tissue | 10 min | 4 hrs | 24 hrs |

T7-lacZ

| | | | |
|---|---|---|---|
| Blood | 2.8 × 10⁹ ± 4.7 × 10⁸ | 3.3 × 10⁶ ± 1.0 ± 10⁵ | 1.1 × 10⁴ ± 1.9 × 10⁴ |
| Brain | 8.7 × 10⁵ ± 3.9 × 10⁵ | 4.3 × 10³ ± 2.1 ± 10³ | 1.1 × 10² ± 1.1 × 10² |
| Lung | 3.8 × 10⁷ ± 6.7 × 10⁶ | 2.5 × 10⁶ ± 2.8 ± 10⁶ | 1.1 × 10⁵ ± 8.1 × 10⁴ |
| Liver | 5.4 × 10⁹ ± 5.5 × 10⁹ | 7.8 × 10⁶ ± 3.9 ± 10⁶ | 8.5 × 10³ ± 3.7 × 10³ |
| Kidney | 4.7 × 10⁷ ± 1.1 × 10⁷ | 2.1 × 10⁶ ± 6.0 × 10⁵ | 5.6 × 10⁴ ± 1.6 × 10⁴ |
| Spleen | 1.2 × 10⁹ ± 2.9 × 10⁸ | 3.3 × 10⁷ ± 2.4 × 10⁷ | 2.5 × 10⁵ ± 8.3 × 10⁴ |

T7-A3

| | | | |
|---|---|---|---|
| Blood | 1.8 × 10⁹ ± 2.4 × 10⁸ | 1.2 × 10⁶ ± 6.1 × 10⁵ | 1.6 × 10⁴ ± 1.7 × 10⁴ |
| Brain | 3.9 × 10⁶ ± 5.7 × 10⁵ | 1.7 × 10⁶ ± 1.3 × 10⁶ | 1.5 × 10⁵ ± 7.7 × 10⁴ |
| Lung | 9.9 × 10⁷ ± 2.7 × 10⁷ | 5.2 × 10⁷ ± 1.7 × 10⁷ | 5.2 × 10⁶ ± 5.2 × 10⁶ |
| Liver | 6.0 × 10⁹ ± 5.1 × 10⁹ | 4.1 × 10⁷ ± 1.2 × 10⁷ | 3.4 × 10⁵ ± 1.9 × 10⁵ |
| Kidney | 1.6 × 10⁸ ± 6.2 × 10⁷ | 5.1 × 10⁷ ± 3.0 × 10⁶ | 3.4 × 10⁶ ± 3.8 × 10⁶ |
| Spleen | 1.7 × 10⁹ ± 3.0 × 10⁸ | 2.6 × 10⁸ ± 2.0 × 10⁷ | 1.9 × 10⁶ ± 1.6 × 10⁵ |

Similar differences are also observed for the T7-A3 phage shortly after administration but with more prolonged circulation times, the relative retention of the T7-A3 phage within brain becomes apparent. This illustrates how a thorough pharmacokinetic analysis against a sufficient number of reference tissues is required before a determination of organ selectivity can be made and the importance of always normalizing to the control phage platform itself. Comparing the ratio of SI values for the putative target organ to the various references organs, i.e., the organ specificity index value, allows a comparison normalized to the control phage content of the two tissues. For example evaluation of the SI value for brain compared to lung at the 4 hour time point provides a value of 288 for brain and 47 for lung, which indicates the relative specificity of the T7-A3 isolate for brain is 6-fold. However, as illustrated in the pharmacokinetic analysis of the test and control reference phage, arbitrarily picking a single time point, especially when plasma levels of phage are high, can be misleading in the evaluation of organ targeting properties.

### B. Effect of Dose on the Brain-Targeting Properties of T7-CAGALCY

Five different doses of T7-A3 phage and T7-lacZ control phage were injected into FVB female mice: 1×10⁷, 1×10⁸, 1×10⁹, 1×10¹⁰, and 1×10¹¹ pfu of each phage. For each dose, three mice were injected. Four hours after phage administration, phage were recovered from the brain, lung, liver, kidney, colon, and blood.

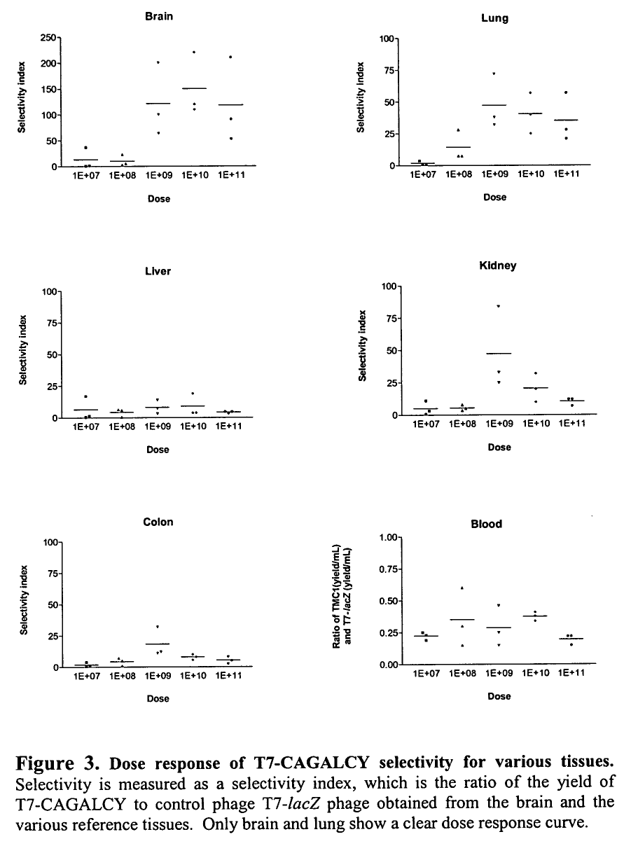

FIG. 3 shows the effect of phage dose on the selectivity index values of the T7-A3 phage for various organs. This was measured as A3-T7 phage yield per gram tissue from various organs normalized by the yield/g tissue of the control phage.

In Tables 2-3 below, values are given for the selectivity index (SI), and the relative tissue selectivity, i.e., the specificity index (SPI). .

As defined above, the selectivity index (SI) given in Table 2 represents the ratio of the yield of the targeting phage, T7-A3 to the yield of control phage, T7-lacZ.

Table 3 shows the organ specificity index (SPI) which represents the ratio of SI values of brain to the various reference tissues.

TABLE 2
Selectivity index values for the brain and various reference tissues: mean ± SEM
values of the data presented in FIG. 3

| | Dose Administered | | | | |
|---|---|---|---|---|---|
| Tissue | 1 × 10⁷ pfu | 1 × 10⁸ pfu | 1 × 10⁹ pfu | 1 × 10¹⁰ pfu | 1 × 10¹¹ pfu |
| Brain | 13.2 ± 11.9 | 10.5 ± 6.3 | 121 ± 40.7 | 150 ± 35.1 | 118 ± 47.3 |
| Lung | 1.9 ± 0.8 | 14.3 ± 6.8 | 47.3 ± 12.5 | 40.7 ± 9.2 | 35.3 ± 11.0 |
| Kidney | 5.0 ± 3.1 | 5.4 ± 1.3 | 47.3 ± 18.5 | 20.7 ± 6.4 | 10.3 ± 1.7 |
| Liver | 6.3 ± 5.4 | 4.2 ± 1.9 | 8.2 ± 3.1 | 8.9 ± 5.1 | 4.2 ± 0.4 |
| Colon | 1.8 ± 1.1 | 4.3 ± 1.8 | 18.3 ± 6.8 | 7.9 ± 1.2 | 5.3 ± 1.6 |

TABLE 3
Organ Specificity Index (SPI) (ratio of SI values of brain to
various reference tissues: mean values)

| Reference | Dose (pfu) | | | | |
|---|---|---|---|---|---|
| Organ | 1 × 10⁷ | 1 × 10⁸ | 1 × 10⁹ | 1 × 10¹⁰ | 1 × 10¹¹ |
| Lung | 4.23 | 0.65 | 3.23 | 4.03 | 3.3 |
| Liver | 2.34 | 3.27 | 26.0 | 23.7 | 28.7 |
| Kidney | 1.92 | 2.08 | 3.27 | 10.4 | 10.8 |
| Colon | 4.57 | 2.82 | 8.87 | 18.7 | 35.5 |

SI values for T7-A3 in brain increased from 13.2 for 1×10⁷ pfu/phage and 10.5 for 1×10⁸ pfu/phage to 121 for 1×10⁹ pfu/phage and 150 for 1×10¹⁰ pfu/phage. Targeting to lung, kidney, liver and colon tissues appears to be dose-dependent in the doses ranging from 1×10⁷ pfu/phage to 1×10⁹ pfu/phage. These changes in SI values were all due to changes in the yield/g tissue of the test A3-T7 phage. The yield/g tissue of the control phage T7-lacZ recovered from the various organs in contrast, was independent of the dose in all the organs examined. These data indicated that the yield/g tissue increased in a dose-dependent manner and the binding of T7-A3 phage to the brain is specific.

SPI values (i.e., ratio of SI) values for brain/liver, brain/kidney, and brain/colon were greater than 10 at doses of 1×10¹⁰ pfu and 1×10¹¹ pfu. SPI value for brain/liver at the 1×10⁹ pfu dose was 26, the only SPI value greater than 10 at this dose. In contrast, the SPI values for lung, liver, kidney or colon compared to the other organs were all lower than 10 at all doses tested.

T7-A3 phage appeared to be retained in the brain throughout the time course examined from 2 min to 24 hr, and only showed a very slow decline after 2 hr post injection. At this time point, the number of T7-A3 phage in the blood had decreased more than 3 logs. Retention of T7-A3 phage in the brain may be the result of both interaction of T7-A3 phage with brain and translocation of T7-A3 phage into the brain tissue

Although, T7-A3 phage was cleared more slowly than T7-lacZ control phage in all tissues examined, T7-A3 phage exhibits the longest tissue half-life in the brain (498 min vs. 43 min for T7-lacZ phage). The half-lives of T7-A3 phage in other tissues are 330, 78, 468, 348 min for the lung, liver, kidney, and spleen respectively. The corresponding half-lives of T7-lacZ in these tissues are 18, 50, 270, 221 min. These data confirm that T7-A3 phage selectively targets the brain tissue.

Targeting of T7-A3 to the brain was dose-dependent. The brain SI values increased significantly from 10 for the 1×10⁸ pfu dose to 121 and 150 for the 1×10⁹ pfu and the 1×10¹⁰ pfu dose respectively. Further increasing the dose to 1×10¹¹ pfu did not increase the SI value, indicating the binding sites in the brain tissue may be saturated at this dose. The binding of T7-A3 phage to the various reference organs also appeared to be dose-dependent at lower doses, but, the SI values are all lower than for brain.

### EXAMPLE 3

### Competition of T7-A3 Phage Brain Biodistribution by Monomeric A3 Fusion Protein

In this example, the ability of the displayed peptide to recapitulate the brain targeting properties outside the context of the T7 phage particle was tested. The first approach was to generate a glutathione (GST)-A3-fusion protein, where the peptide was displayed at the carboxy terminus of GST. The insert encoding the CAGALCY sequence was subcloned into the pGEX-5×-3 vector (Pharmacia Biotech), transfected into the JM 109 host bacteria and individual clones amplified to confirm the construct by sequencing. The BL21 strain was used for protein expression. Overnight cultures were diluted 1:100 in 2×YT broth and incubated at 30° C./250 rpm till the OD₆₀₀ reached 0.5. IPTG was added to the culture to a final concentration of 1 mM to induce GST fusion protein expression and the culture incubated for a further 3 hrs. The cell pellets were suspended in PBS containing 100 μg/mL lysozyme and 10 μg/mL of DNase I. The suspensions were sonicated for 30 min in an ice-water bath. GST fusion proteins in the supernatants were purified using a Glutathione Sepharose 4B gel column according to the manufacturer's instructions (Pharmacia Biotech, but washing the column extensively with 1% detergent buffer to minimize endotoxin contamination). Fusion proteins were eluted with reduced glutathione, and dialyzed against PBS overnight.

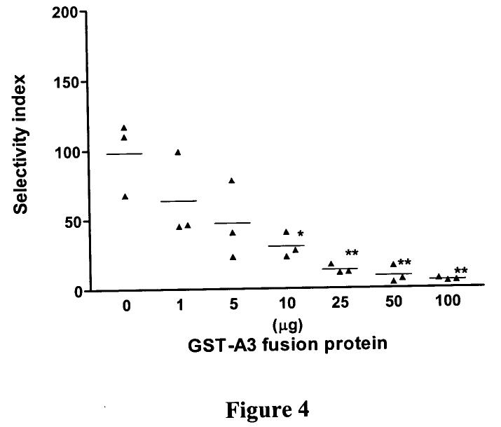

In the first pilot study, groups of 3 mice were dosed with either 50 or 500 μg of GST-A3-fusion or GST control protein followed by administration of 1×10¹⁰ pfu T7-A7 plus T7-lacZ control phage. Tissues were harvested after 2 hours and the content of each phage type determined by plaque analysis. While the GST fusion protein had no effect at either dose on the total yield of T7-A3 phage or SI value, the presence of 50 μg GST-A3 reduced the yield of T7-A3 recovered from brain by 90% and the 500 ug dose reduced the yield of T7-A3 phage to the level of control phage as reflected by SI values of 1. To determine the ED₅₀, a second in vivo competition study was performed where groups of BALB/C mice (n=3 per group) were dosed by intravenous injection with either 0, 1, 5, 10, 25, 50 or 100 μg of GST-A3 fusion protein followed by administration of a mixture of 1×10¹⁰ pfu each of T7-A3 and T7-lacZ control phage. Two hours later the brain tissue was harvested and phage content analyzed. As shown in FIG. 4, the GST-A3 fusion protein was effective at competing for brain tissue binding of the T7-A3 phage. Curve fit analysis indicated that monomeric GST-A3 fusion protein competed binding of the multivalent phage with an apparent ED₅₀ of 450 nM, confirming that the brain targeting activity resided within the displayed A3 peptide. This together with additional information presented below, indicates that the brain targeting activity resides entirely within the displayed peptide, and suggests that a variety of constituents can be substituted for the phage coat protein at the amino terminus without impairing this activity.

Pharmacokinetic analysis showed that while the GST-fusion protein had a plasma t½ of 1 to 2 hours, that of free synthetic peptide was in the order of 1 to 2 minutes. To demonstrate that free synthetic peptide could retain full brain binding activity, the CAGALCY peptide was synthesized by contract by American Peptide Corp. After confirming the peptide sequence and cyclization by GC-MS spectrometry, an in vivo study was performed where 100 ug of peptide was co-injected with 1×10¹⁰ pfu T7-A7 plus T7-lacZ control phage, and then the peptide re-administered at 15 minute intervals to maintain plasma levels. A control peptide lacking the terminal essential tyrosine (see below) was used as a negative control. After 1 hour, the brain tissue was harvested and phage content analyzed by plaque analysis. The results indicated that the free peptide reduced T7-A3 by 90% to levels approaching those of the control T7-lacZ phage, i.e., the free synthetic peptide could also compete for T7-A3 phage binding to brain tissue. Taken together the data demonstrate that the activity of the A3 ligand can be readily transferred to any moiety providing the free aromatic-acid functionality is maintained.

### EXAMPLE 4

### Individual Variants

In this example, various individual peptide variants of A3 were tested in vivo for their ability to target brain or other tissues. For evaluation of individual peptides, 0.5-1×10¹⁰ pfu of each phage was co-injected with a similar number (pfu) of T7-lacZ control phage into 3 FVB or BALB/c mice and after 2 hours of circulation time, the brain, blood and reference organs were harvested. The phage contents of each organ were determined by plaque assay in the presence of X-gal (500 μg/mL) in top agar.

The results of these studies are summarized in Table 4. The selectivity indices given in Table 4 are for brain tissue harvested and analyzed as described in Examples 1 and 2 above. While specific numbers are given, the margin of error is such that it is meaningless to make finely nuanced comparisons. Comparison of actual values from column to column or within a column is tentative and based on an order of magnitude only. Thus, selectively indices of less than 10 are considered low, those between 10 and 30 are considered moderate and those over 100 are considered high.

TABLE 4

| | | | |
|---|---|---|---|
| | Brain | Selectivity | Selectivity |
| Phage (peptide | Targeting | index (FVB | index (BALB/C |
| sequences) | Activity | female) | mice) |

| | | | |
|---|---|---|---|
| CAGALCY | Yes | >200 | 1000 |
| Selected point | | | |
| mutations | | | |
| CAGALCF | Yes | >200 | 800 |
| CAGALC | No | 1.5 | ND |
| CAGALCS | No | 3.0 | ND |
| CAGALCAY | No | 1.4 | ND |
| CAGALSY | No | 1.9 | ND |
| CAGAPCY | No | 7.6 | ND |
| Glycine scan | | | |
| across the ring | | | |
| CGGALCY | No | 0.7 | ND |
| CAGGLCY | No | 0.9 | ND |
| CAGAGCY | No | 1.1 | ND |
| Reducing ring | | | |
| size | | | |
| CGALCY | No | 0.8 | ND |
| CALCY | No | 0.8 | ND |
| CLCY | No | 3.5 | ND |
| Increasing ring | | | |
| size | | | |
| CGADALCY | No | ND | 1 |
| C or N terminal | | | |
| additions | | | |
| CAGALCY-X₍₈₋₁₂₎ | No | ND | ND |
| X₍₁₂₎-CAGALCY | Yes | >200 | ND |
| Known integrin/ | | | |
| disintegrin | | | |
| mimetics | | | |
| CGFDMPC | No | 0.5 | ND |
| CYGDPC | No | 0.8 | ND |
| YCDAPC | No | 0.7 | ND |
| CPADCY | No | 0.3 | ND |
| CRDVNECD | No | 0.3 | 1 |
| CDITEYC | No | 0.4 | 1 |
| CRDVNECDITEYC | No | ND | 2.6 |
| CAGDSMCY | No | ND | 0.9 |

ND: not determined

Table 4 shows that diminution of the ring size whereby less than four amino acids are contained between the two cysteine residues results in a loss of targeting activity.

A systematic substitution of glycines for each non-glycine residue within the ring (glycine scan) was performed and phage expressing the individual peptides A2G, A4G and L5G were evaluated as described in Examples 1 and 2 for targeting to various tissues. All these changes resulted in loss of brain targeting activity.

As shown also in Table 4, addition of substituents at the N-terminus of CAGALCY does not interfere with brain targeting activity. Retention of an aromatic residue at position 7 permits this activity to be retained. However, deletion of this residue, or substitution of it for any amino acid other than an aromatic (tyrosine or phenylalanine) or spacing it from the ring results in loss of activity, as does disruption of ring formation capacity (CAGALSY) disrupting the conformation of the ring by introducing an intracyclic proline kink (CAGAPCY) or decreasing ring size. Further, extending the amino acid sequence at the C-terminus (CAGALCY-X (8-12)) destroys targeting activity indicating the necessity of a free carboxyl group at this position for targeting activity. In general, distorting the general shape of the cyclic peptide results in loss of targeting.

It has been shown that a precise orientation of an tyrosine acid is essential for mimetics of certain integrin binding sites and that it is the precise orientation of this tyrosine acid within the context of the cyclic peptide that confers specificity (Gadek, T. R., et al., Science (2002) 295:1086-1089; Jackson, D. Y., et al., J. Med. Chem. (1997)

-3368; Abraham, D. G., et al., Mol. Pharmacol. (1997) 52:227-236; Blackburn, B. K., et al., J. Med. Chem. (1997) 49:717-729 and Pfaff, M., et al., J. Biol. Chem. (1994) 269:20233-20238). These mimetics were expressed on phage and tested for their ability to target the brain but all proved to be negative, including the mimetic of the αnβ3 integrin antagonist described in U.S. Pat Nos. 5,767,071 and 5,780,426 (CAGDSMCY) which shows a terminal extracyclic free tyrosine at the carboxy terminus.

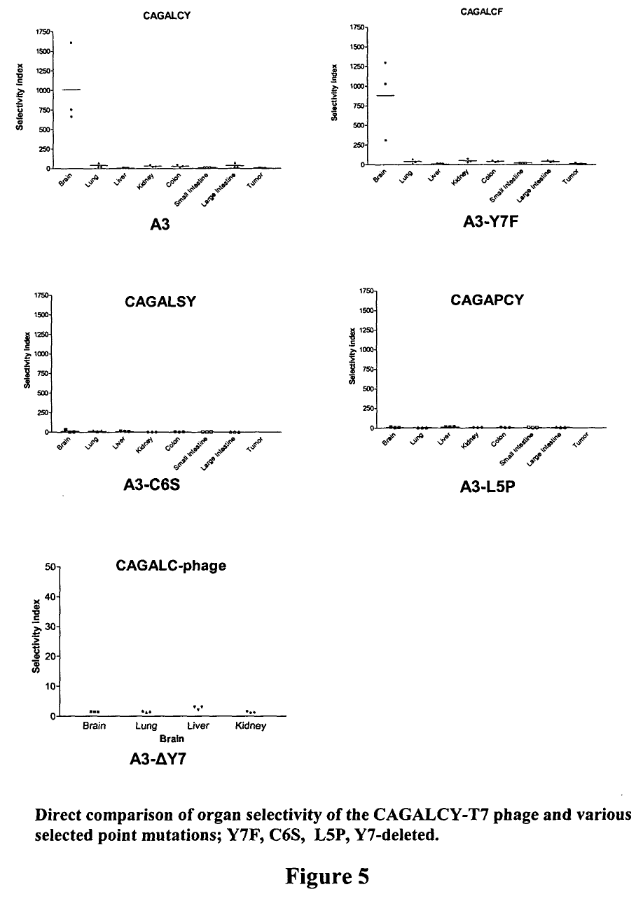

The data obtained for various tissues for a few selected variants are shown in FIG. 5 as an example of the analysis performed on all the various mutations summarized in Table 4.

As shown in FIG. 5, deletion of the tyrosine residue at position 7 destroys the brain targeting activity, since similar peptides lacking this residue fail to have high selectivity indices in brain. When one of the cysteines is changed to serine, thus preventing cyclization (CAGALSY) or when proline is introduced at position 5 (CAGAPCY) brain targeting capacity is lost.

It is clear that the tyrosine residue must be adjacent to the ring, since brain targeting activity is lost for the peptide CAGALCAY which contains an intervening alanine residue. Similarly, substitution of serine for tyrosine at this position results in loss of brain targeting activity (CAGALCS). However, as shown in FIG. 5, substitution of phenylalanine for tyrosine does not appear to abolish brain-targeting activity.

### EXAMPLE 5

### Testing of Mixtures of all 20 Variants of a Single Position

In addition to testing individual peptides, mixtures of peptides in which multiple substitutions at a single amino acid position are reflected were injected simultaneously to test sensitivity of the A3 peptide to such variation.

To determine the optimum amino acid for position 5, all 20 variants at this position were injected. For co-injection of all 20 variants of CAGAXCY phage, individual phage were grown up and titered. Equivalent amounts of each phage type were added to PBS and 200 μL dose of the mixture was injected into 3 BALB/c mice per group (two time points of 30 minutes and 4 hours were studied, total of 6 mice). At 30 min and 4 hrs post injection, the mice were anesthetized, perfused and blood and brain were harvested. Aliquots of both the initial phage pool used for dosing as well as brain tissue samples were plated out, and approximately 100 individual plaques from each set were amplified by PCR using the T7 Super up primer method and sequenced using a CEQ 2000XL sequencer (Beckman Coulter, Calif.).

A total of 107 individual phage from the initial mixture used for dosing were sequenced to verify the relative abundance of the individual phage within the administered dose and these were compared with the relative frequency of the different phage recovered from brain after 30 minutes or 4 hours of circulation. There was a marked and rapid shift in frequency even by 30 minutes, where there was a marked enrichment of phage expressing N at the X5 position, (from 7% in the initial pool to 49%), M (from 5% to 24%) or L (2.8% to 12%) at 30 minutes. By 4 hours, these were the only three phage recovered from brain at a frequency of 55%, 37% and 7.5% for the N, M and L variant, respectively. However, the percentage of phage expressing the original CAGALCY sequence (2.8%) in the input pool was lower than that of the CAGANCY (7.5%) or CAGAMCY (5.6%) phage.

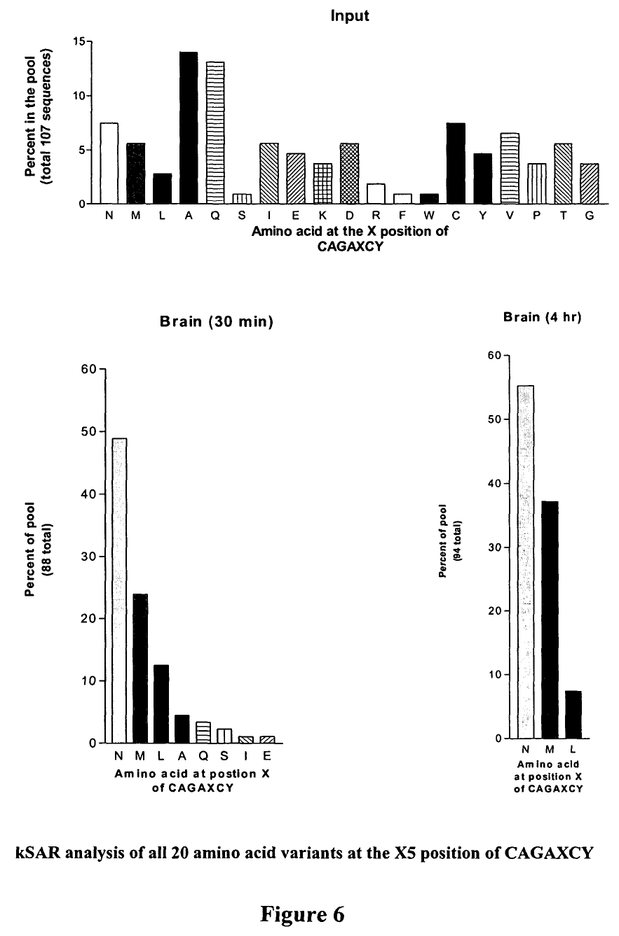

These results are shown in FIG. 6. The prevalence of asparagine at position 5 indicates that hydrophobicity may not be required at this position.

All 20 variants of position 7 were generated and used in a single experiment as described above. Similar to the previous experiment, at 30 min and 4 hrs post injection, blood and brain were harvested, and phage plated. Plaques were randomly picked, the inserts amplified for sequencing and the sequence frequencies compared to an equivalent number of insert sequences obtained from the mixture used for injection.

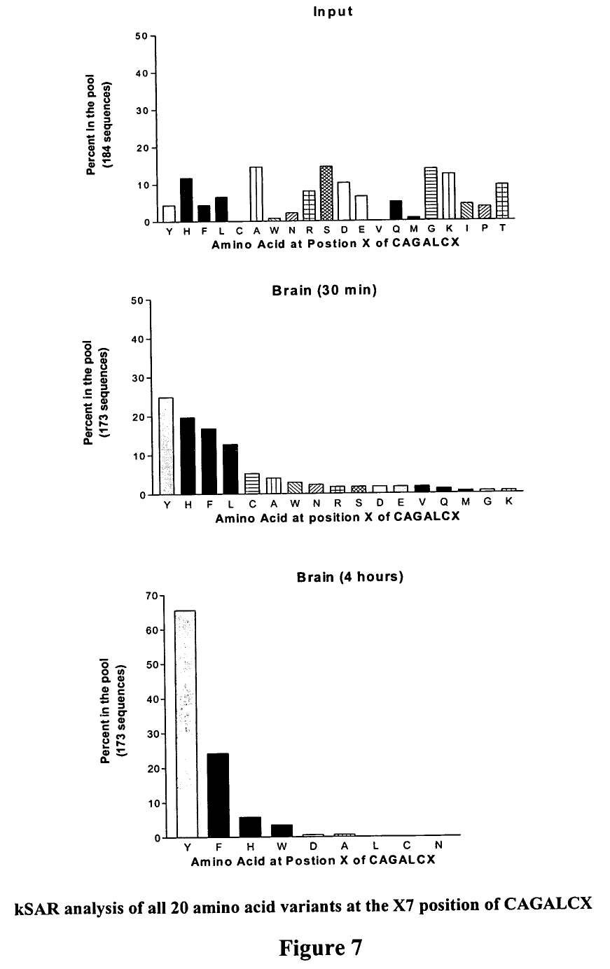

FIG. 7 shows the results. As demonstrated, only the tyrosine and phenylalanine variant showed any accumulation within the brain. All other variants except the histidine and tryptophan variants were rapidly eliminated from the target site. These data indicate that phenylalanine is a reasonably successful substitute for tyrosine at position 7, but no other amino acid in this group is able to replace the tyrosine successfully, and that a carboxy-terminal aromatic residue displaying a free acid is required at this position for brain targeting activity.

### EXAMPLE 6

### Mixtures of Variants at Multiple Positions

A CX₄CY library was prepared and tested. 1×10¹⁰ pfu of the library phage were injected into 6 BALB/c mice (3 mice/time point). At 30 min and 4 hr post-injection, brain was harvested and processed as described above. Similar numbers of plaques were picked individually, and the insert sequence amplified by PCR and sequenced. Alignment analysis of the sequence data was performed with a version of Clustral/W.

Table 5 shows the distribution of peptides recovered. There were no enriched phage with the A3 peptide or with CAGANCY or CAGAMCY within the 83 sequences sampled from the brain at 4 hrs post phage injection. However, phage expressing sequences of CXGXLCY, where X is predominantly valine, were enriched. Since the CXGXLCY motif, where X represents a hydrophobic amino acid, consistent with the structural requirements determined in the previous examples for brain targeting activity, accounts for 57% of phage recovered from the brain, whereas only 2% of the same motif was found in the unselected library, it can be concluded that the selection of brain targeting phage in vivo is highly effective even with just one round of panning.

TABLE 5

| | | |
|---|---|---|
| CAGVLCY---7 | CVGQLCY---7 | CLKDECY---7 |
| CAGVLCY---7 | CVGVLCY---7 | CCASWCY---7 |
| CAGVLCY---7 | CVGSLCY---7 | CISEVCY---7 |
| CAGVLCY---7 | CVGSLCY---7 | CMFEVCY---7 |
| CAGVLCY---7 | CVGSLCY---7 | CRSPVCY---7 |
| CAGVLCSNEV 10 | CVGSMCY---7 | CVVGVCY---7 |
| CAGTLCY---7 | CVGPLCY---7 | CVV-------3 |
| CDGVLCY---7 | CVGPLCY---7 | CVSGVCY---7 |
| CGGVLCY---7 | CVGPSCY---7 | |
| CEGVLCY---7 | CVGDLCY---7 | CXICICD---7 |
| CEGVLCY---7 | CNGDLCY---7 | CLVLPCS---7 |
| CSGVLCY---7 | CVGALCY---7 | CTXLPCVY--8 |
| CSGVLCY---7 | CVGALCY---7 | CTSLICY---7 |
| CPGVGCY---7 | CVGALCY---7 | CCVLVLLVR-9 |
| CRGPLCY---7 | CVTALCY---7 | |
| CTGSLCY---7 | CLGAWCY---7 | CIEGGLLLVR-10 |
| CSGSLCY---7 | CLGALCY---7 | CSGLVLLVR-9 |
| CLGSLCY---7 | CSGALCY---7 | CVGALLLLVR-10 |
| CLGSLCY---7 | CLPPRCY---7 | CSSGCVTSEV-10 |
| CLGPLCY---7 | CIPGSCY---7 | CSGGCVTSEV-10 |
| CLGPLCY---7 | CGLGGCY---7 | CLAGRVTSEV-10 |
| CLGPLCY---7 | CGLAGCY---7 | CVGSCVTSEV-10 |
| CLGVLCY---7 | CR-EAGCY---7 | CVSSCVTSEV-10 |
| CVGELCY---7 | CRYRDCY---7 | CVASGVTSEV-10 |
| CVGNLCY---7 | CPPRPCY---7 | LWGSCVTSEV-10 |
| CVGTLCY---7 | | |
| CVGQLCD---7 | CVELVPCXLV-10 | CW-PCGVSEVS 10 |
| CVGQLCN---7 | CWLPPCY---7 | C--PCGVCY--7 |
| CVGVLCNY--8 | CQLSECY---7 | |

### EXAMPLE 7

### A Comparison of Alternative Candidates Identified by Kinetic SAR Analysis

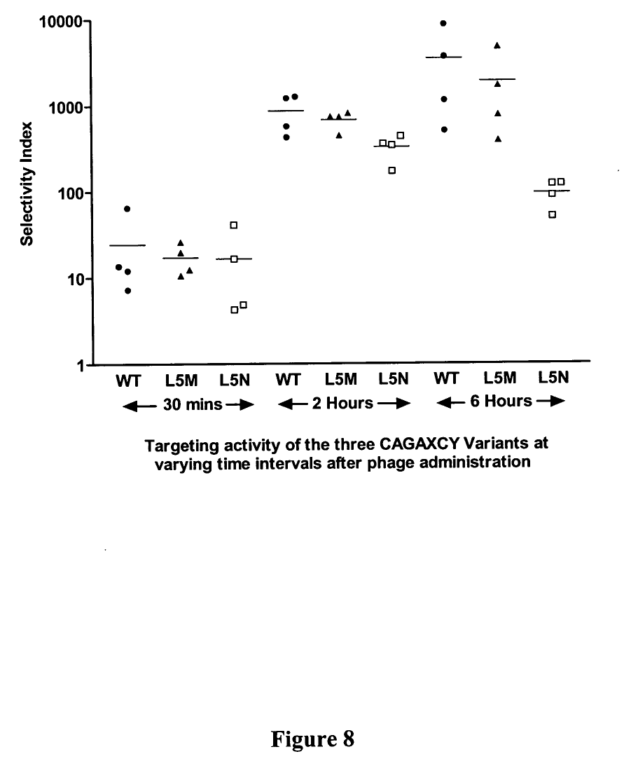

To assess the relative targeting efficiencies of individual mutants shown in FIG. 5 that were enriched along with the parental CAGALCY sequence during the kSAR analysis described in Example 5, compared with the parent CAGALCY-expressing (T7-A3) phage, phage expressing CAGAMCY, CAGANCY, and CAGALCY peptides were amplified, purified and individually evaluated with respect to control lacZ phage in vivo. As shown in FIG. 8, all three phage exhibit a marked increase in SI values for the brain, between 30 minutes and 2 hours. At 2 hours the SI values range from 500 to 1000. The SI values increase further at 6 hours after administration for the parent CAGALCY and M5 mutant but show a slight decrease for the N5 mutant. These results confirm that the enrichment of these three variants at the 5 position after administration of the multiple variant mixture that was observed in the study presented in Example 5 is predictive of their targeting activity when validated against the control phage.

### EXAMPLE 8

### Molecular Modeling

The results set forth above provide the basis for delineating structure activity relationships and the construction of molecular models which confirm the space/charge/hydrophobicity conformation required for activity. To construct these models, Alchemy 2000®, a molecular modeling software from Tripos, Inc. was used. This package contains four different options for minimizing molecular structures:

1. the SYBYL Molecular Mechanics Force Field;

2. the MM3 Molecular Mechanics Force Field;

3. the PM3 Semi-Empirical Molecular Orbital Hamiltonian, highly parameterized for minimization of three dimensional structures; and

4. the MOPAC Technique, a semi-empirical molecular orbital methodology optimized for calculating heat of formation energies for small to medium size molecular systems.

Initial models based on the linear zwitterionic form of A3 using SYBYL showed a preference for a conformation where the sulfhydryl groups of the cysteines are at a typical SS bond distance; the PM3 Hamiltonian technique showed that the cyclized form is considerably more stable than the acyclic form. While these results explain the importance of retaining a carboxylic acid group proximal to the cycle in that it predicts the formation of a salt bridge between the NH₃⁺ and COO⁻ groups at the N- and C-termini, this feature appears to be unlikely, since protonation of the N-terminal group is unlikely in the context of phage display where the A3 peptide is coupled at its N-terminus to the phage coat protein.

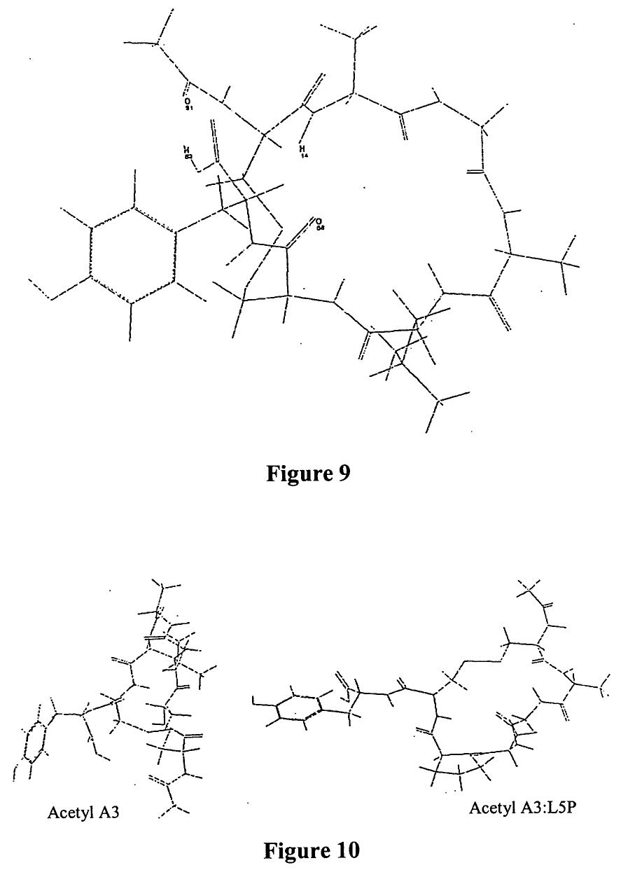

Therefore, the linear form which was modeled by the PM3 methodology was used as a starting point but with the C-terminal group converted to COOH and the N-terminal group was acetylated (NHCOCH₃). The PM3 methodology was then used to minimize the structure and create a cyclic form. The resultant is shown in FIG. 9 and predicts two intramolecular hydrogen bonds—one between H88 of the terminal carboxylic group and the 091 of the carbonyl group of the backbone amide bond and the other between H14 and O58.

The requirement for the space/charge/hydrophobicity relationship shown in FIG. 9 explains the failure of the A3 variant L5P to display targeting activity. The structure of the cyclic acetylated form of this variant was minimized using the PM3 Hamiltonian, and compared to the corresponding structure of cyclic acetylated A3 as shown in FIG. 10. As shown, the orientation of the phenyl ring is distorted in the variant, and the hydrogen bond between O91 and H88 is lost; and the position of the terminal COOH is shifted relative to the ring.

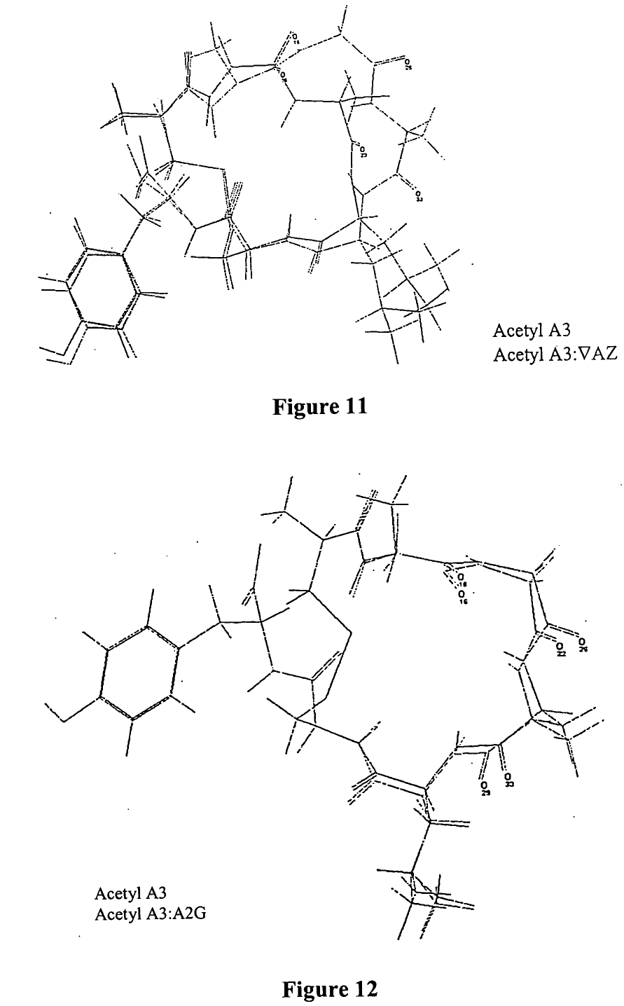

A comparison of the space/charge/hydrophobicity conformation of the acetyl form of a cyclic analog with diminished ring size, CGALCY, is compared with acetyl cyclic A3 in FIG. 11. The PM3 Hamiltonian modeling technique indicates that the structure of CX₃C does not favor the formation of a stable cyclic peptide.

FIG. 12 shows a comparison similar to that of FIGS. 10 and 11 for acetyl cyclic A3 with acetyl cyclic A3 analog A2G. While these structures appear similar, as shown above, this variant fails to exhibit brain targeting capability.

### EXAMPLE 9

### A3 and Functional Derivatives Function as Anti-inflammatory Agents

Based on the molecular modeling of the CAGALCY sequence of A3 and the absolute requirements for an aromatic residue at position 7 with a free terminal carboxylic acid, and the preferred embodiments of the remaining chemical 3D pharmacophore for brain targeting activity, the prediction was that this ligand represented a novel member of the integrin ligand mimetic superfamily with high specificity for brain. In this example the functional utility of the A3 and preferred derivatives is demonstrated in two inflammatory models where brain inflammation plays a key role in the pathogenesis of disease: the lipopolysaccharide-induced model of septic shock, and the Plasmodium berghei murine model of cerebral malaria. In both diseases, an excessive inflammation coupled with aberrant disseminated intravascular coagulation (DIC) lead to shock with a high risk of mortality. The effect of the A3 ligand mimetic on blood cell adhesion to the brain microvasculature is described in paragraph A below. The effect of A3 on the clinical outcome of these severe inflammatory diseases is outlined in paragraph B.

The effect of the A3 mimetic of blood cell adhesion to brain vasculature was assessed by intravital microscopy. Platelets or leukocytes were fluorescently labeled and then the interaction of the labeled cells with the vascular endothelium imaged and analyzed pre and post the addition of various compounds. Platelets were isolated from blood of source animals by differential centrifugation at 200 g for 10 minutes, to pellet erythrocytes and leukocytes. The supernatant, designated platelet-rich plasma contained less than 0.5% leukocytes as determined by the number of DAPI-positive nucleated cells. The platelets were pelleted by spinning at 500 g, resuspended in Tyrode's buffer and incubated for 20 min with 5-,-6-carboxyfluorescein diacetate, succinimidyl ester (CFDA, or CFSE). 1×10⁸ platelets in 0.1 ml were injected slowly over 5 minutes via a carotid catheter into anesthetized mice. The body temperature was maintained at 37° C. by a heating pad with a rectal thermometer. Platelet rolling and adhesion was recorded for off-line analysis onto videotape or onto hard-drive of the computer via an intensified camera (Cascade, Roper Scientific) on a Leica DM-FSA upright microscope. The pial vessels were visualized by surgically developing a small window in the skull. Leukocytes are fluorescence-labeled in vivo by injecting Rhodamine 6G via a carotid catheter and analyzing the movement of the labeled cells on brain endothelium.

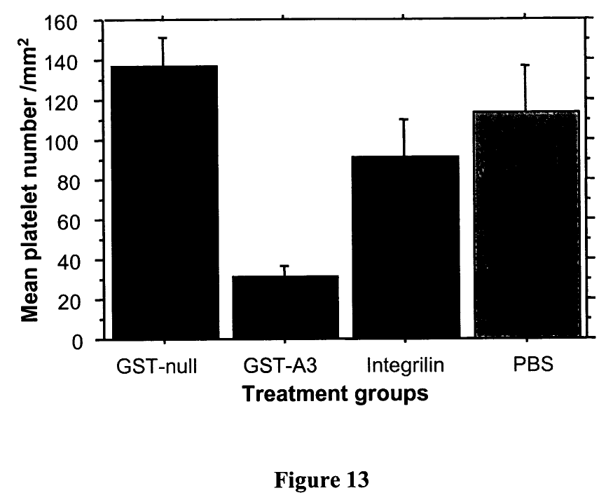

Intraperitoneal injection of 10 μg of LPS significantly increases platelet and leukocyte rolling and adhesion in pial vessels with the peak response occurring 4 hours after injection. Therefore this timepoint was chosen to evaluate the adhesion of differentially labeled platelets or leukocytes to the pial vessels in the presence of a fusion protein of the CAGALCY ligand or GST-null control protein. For the platelet studies a known antagonist of platelet-platelet interactions, Integrilin™ (Cor Therapeutics) was also evaluated. For the labeled leukocyte studies, a neutralizing anti-ICAM-1 antibody was used. As shown in FIG. 13, the fusion protein expressing the CAGALCY ligand effectively inhibited platelet adhesion in this DIC model of sepsis (P<0.0001). In contrast, the GST control protein had no effect, and Integrilin, administered at the clinical dose per mouse (40 mg/kg), showed a slight antagonistic effect but this was not statistically significant. Indeed at higher doses Integrilin exacerbated DIC and adhesion of microaggregates to the brain vasculature. The A3-GST fusion protein had no effect on the adhesion of labeled leukocytes to the pial vessels. Only the anti-ICAM-1 antibody was able to completely reverse leukocyte adhesion.

P. berghei infection of mice results in marked platelet and leukocyte adhesion in the pial vessels on day-6 of infection the time point when infected mice develop impaired consciousness and are moribund. Similar effects of the GST-A3 fusion protein but not the GST-null protein on blocking platelet and microthrombi adhesion to the pial vessels were observed.

B. The effect of the A3-fusion protein on survival in the LPS-induced sepsis model was then assessed. Three groups of mice (n=10 per group) were injected i.p. with an LD₅₀ dose of LPS (150 micrograms). One group received no drug, one group received the control vehicle and one group received the A3-fusion protein (500 micrograms, 2 times per day). The mortality rates were 60% in the controls groups and only 10% in the A3-fusion protein treated group. All remaining animals recovered by Day 5. These results illustrate how the anti-platelet adhesion effect of the A3 ligand can affect the downstream clinical sequelae of thrombus adhesion within the brain microvasculature that lead to death.

### EXAMPLE 10

### A3 Functions as an Antithrombotic

One well established model for assessing efficacy of anti-thrombotic agents is the ferric chloride (FeCl₃) induced thrombosis model. In this study, a 5% Fe Cl₃ solution was applied to the lower surface of a carotid artery, to which a modified flow probe had been attached. Flow was monitored at 1 minute intervals and time to occlusion measured using a Transonic System. Groups of 6 mice were treated with either GST control protein or the GST-A3 fusion protein at a dose of 1.25 mg/kg just before applying the FeCl solution. Treatment with GST-A3 fusion protein significantly (P<0.05) prolonged time to occlusion in the carotid artery following FeCl₃ treatment compared to PBS controls and GST control protein dosed mice: from 8 minutes (PBS and GST control) to 16-18 minutes (GST-A3). This indicates that the TMC-A3 peptide has anti-thrombotic activity.

1. A method to validate the ability of a compound to target selectively an organ or tissue which method comprises assessing a pharmacokinetic profile of the compound in a model mammalian or avian subject in vivo wherein said pharmacokinetic profile comprises:
(a) a selectivity index (SI) with respect to a non-targeting molecule;
(b) a specificity index (SPI) with respect to other organs or blood;
(c) a comparison of clearance rate from said tissue or organ as compared to clearance rate from blood using a ratio of areas under the curve and
(d) clearance rate from blood relative to the clearance rate from the target organ, as compared to the behavior of a non-targeting molecule where the clearance rates from blood and the target organ are similar and optionally
(e) dose responsiveness wherein SI is assessed as a function of increasing dose; according to the parameters
(a′) the SI is >5
(b′) the SPI is >5
(c′) the clearance rate from said tissue or organ as compared to clearance rate from blood has a ratio of areas under the curve of >5
(d′) faster clearance rate from blood than from the tissue or organ in comparison with the behavior of a non-targeting molecule,
(e′) SI is increased with increasing dose,
whereby a compound which satisfies any three of the parameters (a′)-(d′) is validated as having said ability.

2. The method of claim 1, wherein said compound satisfies all of (a′)-(d′).

3. The method of claim 1, wherein said compound satisfies (e′).

4. The method of claim 1, wherein said compound is a peptide or peptidomimetic.

5. The method of claim 4, wherein the peptide is an antibody fragment.

6. The method of claim 4, wherein the peptide or peptidomimetic is coupled to a retrievable tag.

7. The method of claim 1, wherein said compound is coupled to a retrievable tag.

8. The method of claim 7, wherein the retrievable tag is a phage.

9. The method of claim 7, wherein said retrievable tag is a radioisotope, fluorescent moiety, or magnetic beads.

10. A method to establish the relationship between structure and activity with respect to targeting selectively an organ or tissue for a compound comprising at least one structural feature, which method comprises,
administering to a mammalian or avian subject
(a) a compound containing a single variant of a single structural feature; or
(b) a mixture of compounds containing multiple variants of a single structural feature; or p1 (c) a compound containing a single variant of each of more than one structural feature; or
(d) a mixture of compounds containing multiple variants of each of more than one structural feature; and
assessing the targeting ability of each compound containing one or more variant at various time points.

11. The method of claim 10, wherein said administering is of a compound containing a single variant of said structural feature or are peptidomimetics.

12. The method of claim 10, wherein said administering is of a mixture of compounds containing multiple variants of a single structural feature.

13. The method of claim 10, wherein said administering is of a compound with one variant of each of more than one structural feature.

14. The method of claim 10, wherein said administering is of a mixture of compounds with multiple variants of each of more than one structural feature.

15. The method of claim 10, wherein said compounds are peptides and said structural features are amino acids.

16. The method of claim 15, wherein the peptides are Fv regions.

17. The method of claim 10, wherein said assessing comprises determining the selectivity index of each said compound containing one or more variants, and/or wherein said assessing further includes determining the specificity index of each said compound containing one or more variants.

18. The method of claim 10, wherein said compounds containing one or more variants are coupled to a retrievable tag.

19. The method of claim 18, wherein the retrievable tag is a radioisotope, a fluorescent moiety, or magnetic beads.

20. The method of claim 11, wherein the peptides are administered with a retrievable tag.

21. The method of claim 20, wherein said retrievable tag is a phage.

22. The method of claim 10, wherein said target organ or tissue is brain or lung.

23. The method of claim 10, wherein the target tissue is angiogenic vessels.

24. A compound of the formula
Z—A¹X¹X²X³X⁴A²X⁵ (1)
wherein Z represents a non-interfering substituent;
X¹-X⁵ represent independently selected amino acids; and
wherein each of A¹ and A² represents a cyclizing moiety wherein A¹ and A² are coupled by a covalent bond, and
wherein said compound selectively targets a tissue or organ.

25. The compound of claim 24, wherein each of A¹ and A² is cysteine or homocysteine and are coupled by a disulfide bond.

26. The compound of claim 24, wherein X⁵ is an aromatic amino acid.

27. The compound of claim 24, wherein Z comprises a label.

28. The compound of claim 24, wherein Z is H or comprises a peptide sequence or a linker.

29. The compound of claim 24, wherein X⁴ is a hydrophobic amino acid.

30. The compound of claim 24, wherein X⁴ is leucine, methionine or asparagine.

31. The compound of claim 30, wherein X⁵ is an aromatic amino acid.

32. The compound of claim 31, wherein X⁵ is phenylalanine, tyrosine or m-tyrosine.

33. The compound of claim 32, wherein Z is H or a peptide.

34. The compound of claim 33, wherein the peptide is a single chain antibody, the Fc domain of an antibody, or fragment thereof.

35. The compound of claim 33, wherein Z is H or a peptide of no more than 15 amino acids.

36. The compound of claim 25, wherein X¹-X³ are hydrophobic aliphatic amino acids, X⁴ is N, M or L and X⁵ is Y or F.

37. The compound of claim 36, wherein each of X¹-X³ is independently A, G, L, M, V or I.

38. The compound of claim 37, wherein X¹-X³ are independently A or G.

39. The compound of claim 38, which is Z-CAGALCY.

40. A conjugate comprising the compound of claim 36 coupled to a brain-therapeutic moiety.

41. A method to deliver a therapeutic moiety to the brain, which method comprises administering to a subject the conjugate of claim 40.

42. A method to treat an inflammatory condition, which method comprises administering to a subject in need of such treatment an effective amount of the compound of claim 36.

43. The method of claim 42, wherein said compound is coupled to a vehicle effective to extend in vivo half-life.

44. The method of claim 43, wherein said vehicle is a pharmaceutically acceptable polymer, a peptide, or a delivery vehicle.

45. The method of claim 44, wherein said delivery vehicle is a liposome.

46. A formulation of the compound of claim 36 that increases its vascular retention and/or decreases its clearance and blood half-life.

47. A compound that binds selectively to brain or lung in vivo, which compound comprises at least a portion having the space/charge/hydrophobicity conformation of the compound CAGALCY in cyclic form.

48. A method to target brain or lung which method comprises administering to a subject the compound of claim 36 or a pharmaceutical or veterinary composition thereof.

49. A method to target brain or lung which method comprises administering to a subject the compound of claim 47 or a pharmaceutical or veterinary composition thereof.

50. A method to prepare a library of cyclic polypeptides which method comprises expressing a multiplicity of nucleotide sequences wherein said nucleotide sequences are comprised of codons that putatively encode a peptide of the formula CXₙC with optional extensions at the N- and/or C-terminus,
wherein C is cysteine and X is any amino acid and wherein a codon represented as encoding X may be a termination codon;
n is 2-25;
and wherein the nucleotide sequence encoding Xₙ is sufficiently degenerate that said codons in some cases encode cysteine and in some cases are termination codons;
whereby a multiplicity of polypeptides is produced.

51. The method of claim 50, wherein n is 15 or less, and the putatively encoded peptide does not comprise extensions.

52. The method of claim 51, wherein n is 10.

53. The method of claim 50, wherein the nucleotide sequence corresponding to Xₙ is completely random.

54. The method of claim 50, wherein said polynucleotide is produced from phage.

55. The method of claim 54, wherein said peptides are produced as C-terminal extensions of a phage protein.

56. The method of claim 55, wherein the phage protein is a T7 coat protein.

57. The method of claim 55, wherein said method further comprises cleaving said peptides from said phage protein.
